SEQUENCE LISTING (4 sequences)

SEQ ID NO: 1 - protein, 473 aa, Zymomonas mobilis
MSSESSQGLVTRLALIAAIGGLLFGYDSAVIAAIGTPVDIHFIAPRHLSATAAASLSGMVVVAVLVGCVTGSLLSGWIGIRFGRRGGLLMSSICFVAAGFGAALTEKLFGTGGSALQIFCFFRFLAGLGIGVVSTLTPTYIAEIAPPDKRGQMVSGQQMAIVTGALTGYIFTWLLAHFGSIDWVNASGWCWSPASEGLIGIAFLLLLLTAPDTPHWLVMKGRHSEASKILARLEPQADPNLTIQKIKAGFDKAMDKSSAGLFAFGITVVFAGVSVAAFQQLVGINAVLYYAPQMFQNLGFGADTALLQTISIGVVNFIFTMIASRVVDRFGRKPLLIWGALGMAAMMAVLGCCFWFKVGGVLPLASVLLYIAVFGMSWGPVCWVVLSEMFPSSIKGAAMPIAVTGQWLANILVNFLFKVADGSPALNQTFNHGFSYLVFAALSILGGLIVARFVPETKGRSLDEIEEMWRSQK

SEQ ID NO: 2 - protein, 324 aa, Zymomonas mobilis
MEIVAIDIGGTHARFSIAEVSNGRVLSLGEETTFKTAEHASLQLAWERFGEKLGRPLPRAAAIAWAGPVHGEVLKLTNNPWVLRPATLNEKLDIDTHVLINDFGAVAHAVAHMDSSYLDHICGPDEALPSDGVITILGPGTGLGVAHLLRTEGRYFVIETEGGHIDFAPLDRLEDKILARLRERFRRVSIERIISGPGLGNIYEALAAIEGVPFSLLDDIKLWQMALEGKDNLAEAALDRFCLSLGAIAGDLALAQGATSVVIGGGVGLRIASHLPESGFRQRFVSKGRFERVMSKIPVKLITYPQPGLLGAAAAYANKYSEVE

SEQ ID NO: 3 - protein, 1174 aa, Escherichia coli
MITIDGNGAVASVAFRTSEVIAIYPITPSSTMAEQADAWAGNGLKNVWGDTPRVVEMQSEAGAIATVHGALQTGALSTSFTSSQGLLLMIPTLYKLAGELTPFVLHVAARTVATHALSIFGDHSDVMAVRQTGCAMLCAANVQEAQDFALISQIATLKSRVPFIHFFDGFRTSHEINKIVPLADDTILDLMPQVEIDAHRARALNPEHPVIRGTSANPDTYFQSREATNPWYNAVYDHVEQAMNDFSAATGRQYQPFEYYGHPQAERVIILMGSAIGTCEEVVDELLTRGEKVGVLKVRLYRPFSAKHLLQALPGSVRSVAVLDRTKEPGAQAEPLYLDVMTALAEAFNNGERETLPRVIGGRYGLSSKEFGPDCVLAVFAELNAAKPKARFTVGIYDDVTNLSLPLPENTLPNSAKLEALFYGLGSDGSVSATKNNIKIIGNSTPWYAQGYFVYDSKKAGGLTVSHLRVSEQPIRSAYLISQADFVGCHQLQFIDKYQMAERLKPGGIFLLNTPYSADEVWSRLPQEVQAVLNQKKARFYVINAAKIARECGLAARINTVMQMAFFHLTQILPGDSALAELQGAIAKSYSSKGQDLVERNWQALALARESVEEVPLQPVNPHSANRPPVVSDAAPDFVKTVTAAMLAGLGDALPVSALPPDGTWPMGTTRWEKRNIAEEIPIWKEELCTQCNHCVAACPHSAIRAKVVPPEAMENAPASLHSLDVKSRDMRGQKYVLQVAPEDCTGCNLCVEVCPAKDRQNPEIKAINMMSRLEHVEEEKINYDFFLNLPEIDRSKLERIDIRTSQLITPLFEYSGACSGCGETPYIKLLTQLYGDRMLIANATGCSSIYGGNLPSTPYTTDANGRGPAWANSLFEDNAEFGLGFRLTVDQHRVRVLRLLDQFADKIPAELLTALKSDATPEVRREQVAALRQQLNDVAEAHELLRDADALVEKSIWLIGGDGWAYDIGFGGLDHVLSLTENVNILVLDTQCYSNTGGQASKATPLGAVTKFGEHGKRKARKDLGVSMMMYGHVYVAQISLGAQLNQTVKAIQEAEAYPGPSLIIAYSPCEEHGYDLALSHDQMRQLTATGFWPLYRFDPRRADEGKLPLALDSRPPSEAPEETLLHEQRFRRLNSQQPEVAEQLWKDAAADLQKRYDFLAQMAGKAEKSNTD

SEQ ID NO: 4 - protein, 582 aa, Saccharomyces cerevisiae
MVQESRPGSVRSYSVGYQARSRSSSQRRHSLTRQRSSQRLIRTISIESDVSNITDDDDLRAVNEGVAGVQLDVSETANKGPRRASATDVTDSLGSTSSEYIEIPFVKETLDASLPSDYLKQDILNLIQSLKISKWYNNKKIQPVAQDMNLVKISGAMTNAIFKVEYPKLPSLLLRIYGPNIDNIIDREYELQILARLSLKNIGPSLYGCFVNGRFEQFLENSKTLTKDDIRNWKNSQRIARRMKELHVGVPLLSSERKNGSACWQKINQWLRTIEKVDQWVGDPKNIENSLLCENWSKFMDIVDRYHKWLISQEQGIEQVNKNLIFCHNDAQYGNLLFTAPVMNTPSLYTAPSSTSLTSQSSSLFPSSSNVIVDDIINPPKQEQSQDSKLVVIDFEYAGANPAAYDLANHLSEWMYDYNNAKAPHQCHADRYPDKEQVLNFLYSYVSHLRGGAKEPIDEEVQRLYKSIIQWRPTVQLFWSLWAILQSGKLEKKEASTAITREEIGPNGKKYIIKTEPESPEEDFVENDDEPEAGVSIDTFDYMAYGRDKIAVFWGDLIGLGIITEEECKNFSSFKFLDTSYL

(19) United States
(12) Patent Application Publication
(10) Pub. No.: US 2023/0399672 A1
(43) Pub. Date: Dec. 14, 2023

(54) GENOME-MODIFIED BACTERIAL STRAINS FOR LARGE SCALE BIOPROCESSES

(71) Applicants: University of Stuttgart, Stuttgart (DE); MANUS BIO INC., Cambridge, MA (US)

(72) Inventors: Martin ZIEGLER, Stuttgart (DE); Ralf TAKORS, Stuttgart (DE); Joana SIEMEN, München (DE); Michael LÖFFLER, Öpfingen (DE); Christine Nicole S. SANTOS, Waltham, MA (US); Ajikumar Parayil KUMARAN, Waltham, MA (US)

(21) Appl. No.: 18/034,120

(22) PCT Filed: Nov. 2, 2021
(86) PCT No.: PCT/US21/57657

§ 371 Date: Apr. 27, 2023

Related U.S. Application Data
(60) Provisional application No. 63/108,656, filed on Nov. 2, 2020.

(51) Int. Cl. C12P 21/02; C12N 15/52; C12N 15/70; C12N 9/02; C12N 9/10; C12N 9/12; C12N 9/90

(52) U.S. Cl. CPC C12P 21/02; C12N 15/52; C12N 15/70; C12N 9/0008; C12Y 102/07001; C12N 9/1051; C12Y 204/01; C12N 9/1205; C12Y 207/01002; C12Y 207/01032; C12N 9/90; C12Y 504/99009

(57) ABSTRACT

The present invention in various aspects and embodiments provides engineered bacterial strains having a modified genome that provides advantages in maintenance and biosynthetic processes when cultured at large scale. The present invention further provides methods for large-scale fermentation using the bacterial strains. In various embodiments, the strain engineering yields a reduction in cellular responses to micro-environmental stimuli imposed in large scale bioreactors.

### BACKGROUND

Innovative bacterial strains for biosynthesis of desired chemicals are cultivated in preliminary lab bioprocesses, and must be adapted for large-scale bioprocesses to realize their potential. However, bioprocesses developed in the lab often show deteriorated titers, rates, and yields (or TRY values) when transferred to large-scale industrial conditions. For example, typical large scale processes show lower growth, increased by-product formation, and reduced substrate-to-product formation, which in turn reduces biomass specific productivities, space-time yields, product titers, and purities. Problems of microbial scale-up can be due to insufficient mixing and formation of gradients of dissolved gases, substrates, pH etc. These impose substrate limitations and frequent stress on fluctuating cells.

Engineered bacterial strains exhibiting advantages in maintenance and/or product biosynthesis at large scale culture are desired.

### SUMMARY OF EMBODIMENTS

The present invention in various aspects and embodiments provides engineered bacterial strains having a modified genome that provides advantages in maintenance and biosynthetic processes when cultured at large scale. The present invention further provides methods for large-scale fermentation using the bacterial strains. In various embodiments, the strain engineering yields a reduction in cellular responses to micro-environmental stimuli imposed in large scale bioreactors.

For example, in large-scale fed-batch production processes bacterial cells are exposed to heterogeneous substrate availability caused by long mixing times. Bacterial cells, when moving transiently through nutrient poor zones, react by looping accumulation of the alarmone ppGpp and energetically wasteful cellular responses, resulting in growth and productivity limitations. In various aspects and embodiments of this disclosure, these limitations are ameliorated.

In one aspect, the invention provides a genome-modified bacterial strain that reduces energetically wasteful cellular responses during large-scale culture by deletion or inactivation of non-essential genes. Without being bound by theory, it is believed that the deletions or inactivations avoid costly and unnecessary DNA replication, RNA synthesis, protein synthesis, and/or other ATP-intensive cellular processes during large scale culture. Such gene deletions or inactivations include those coding for flagella components or transcriptional regulators thereof, chemotaxis proteins and regulators thereof, inhibitors of DNA replication, and proteins involved in active transport of sugars other than glucose, among other cellular processes disclosed herein. According to this aspect, the strain exhibits a lower cost of maintenance and higher biosynthesis performance under large-scale conditions as compared to a parent strain that does not contain said deletions or inactivations.

Various bacterial strains may be employed according to this disclosure. In various embodiments, the bacterial strain is a strain of Escherichia coli. In various embodiments, the large-scale conditions are at least about 1000 L, or at least about 10,000 L, or at least about 40,000 L, or at least about 100,000 L.

In various embodiments, the bacterial strain has deletions or inactivations of genes encoding flagella components or transcriptional regulators thereof. For example, in some embodiments, the bacterial strain is a strain of E. coli, and deleted or inactivated genes encoding flagella components or transcriptional regulators thereof are selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, and flhB. In various embodiments, one or more of such genes may be substantially deleted, or promoters deleted or inactivated, or necessary transcription factors deleted or inactivated (at the DNA or protein level), to thereby avoid costly DNA, RNA, and/or protein synthesis. In some embodiments, translation of one or more flagella components can be prevented (e.g., by gene deletion, promoter deletion or inactivation, RBS deletion or inactivation, or protein mutation), thereby avoiding functional flagella assembly and thus avoid loss of ATP via active cell motility. In various embodiments, one or more operons encoding flagella components are deleted, or operon expression is otherwise reduced or eliminated.

In these or other embodiments, the strain comprises deletions or inactivations of one or more genes encoding chemotaxis proteins and regulators thereof. For example, the strain may comprise deletions or inactivations of one or more of E. coli genes tar, tap, cheR, cheB, cheY, cheZ, cheW, and cheA. In various embodiments, one or more of such genes may be substantially deleted, or promoters deleted or inactivated, or necessary transcription factors deleted or inactivated (at the DNA or protein level), to thereby avoid costly DNA, RNA, and/or protein synthesis. In various embodiments, one or more operons encoding such genes are deleted, or expression of the operon otherwise reduced or eliminated.

In some embodiments, the strain has a deletion or inactivation of one or more genes involved in active transport of sugars, especially sugars other than glucose. Exemplary genes according to these embodiments include one or more of E. coli gatA, gatB, gatC, gatD, gatR, and uhpT (or orthologs thereof). In various embodiments, one or more of such genes may be substantially deleted, or promoters deleted or inactivated, or necessary transcription factors deleted or inactivated (at the DNA or protein level), to thereby avoid costly DNA, RNA, and/or protein synthesis. In various embodiments, one or more operons encoding such genes are deleted, or expression of the operon otherwise reduced or eliminated.

Thus, in some embodiments, the bacterial strain comprises deletions or gene inactivations selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

In exemplary strains in accordance with embodiments described above, the bacterial strain is a strain of E. coli comprising deletions or gene inactivations selected from fliC, aroF, aldA, cstA, aceA, cspD, aceB, trg, groL, dnaK, yfiA, gatC, flgL, flgK, acs, mdh, kgtP, fliA, glnH, yjdA, rpoS, hupB, clpB, uhpT, clpA, yeeI, htpG, motA, argT, rplP, flxA, fadD, pheT, flgM, arcA, modA, leuD, fumA, yjcZ, gpmA, rpoE, rseA, maeB, katG, ychH, hns, ygaT, cheW, ybhQ, atpC, glyS, fadB, osmE, hcaR, uspE, uspA, dps, ygeV, pheA, yfiQ, ibpA, ihfB, livK, miaA, ihfA, cysA, bolA, ydcS, ucpA, uspF, nuoH, yeaA, yjjY, aldB, yeaG, araC, yqfA, grpE, aroP, sra, ygjG, cheY, acrB, tpx, cysN, yaeH, osmC, ugpB, ykfB, Smg, yfcY, hypD, elaB, rsd, ybaL, fadH, ubiA, ygaU, sseA, yccV, bfr, pheS, yihX, ytfQ, putP, cycA, yeaT, yiiU, ycgB, rrmJ, rob, agp, ybeL, lrp, ydhR, malP, fbaB, msyB, glcC, ydcJ, ubiC, lon, glpK, dacC, yqjC, glmM, ompR, selD, ygdH, trpA, mukB, nupG, gltD, uspG, yggG, yrbL, glcD, cfa, phoH, yniA, gst, mscL, yhiO, ybhL, yahO, yjgD, glpF, wbbK, yfcX, yqjD, paaJ, yqjE, ynjH, aroL, galS, fruA, mhpR, rnr, prlC, smf, ygaM, ynhG, glgC, csiE, yaiZ, ygiN, aphA, ivy, glgS, ybjP, puuD, udp, yceP, fxsA, ybbN, yhbT, yeaY, ybeZ, ycjG, erfK, yhaO, yjdC, astC, sbmC, yghZ, ydbJ, uxuA, yhaL, fucO, ygdR, yajO, elbB, yhdW, ytfJ, ygfJ, ycfP, yfbT, phoU, blc, pepT, yqcA, yegP, yghU, sfsA, ysgA, yohC, mgtA, ybbK, nhaA, adhP, yehZ, rmuC, ydeW, yqeC, yecA, mppA, treA, dkgA, malS, ddlB, yafY, exuR, yhjY, yfbU, nadR, ydgD, qor, slt, yjgB, yaiE, soxS, prpR, yeiE, yahK, ydjF, yjfO, talA, yodD, pmrD, yhdH, yliB, yliJ, yeeZ, yaeP, ybdK, msrA, ydhQ, rfaB, ybjQ, ygiE, grxB, yjjM, ydhF, ycbB, araF, ygjR, yqjG, cdaR, ymgE, glnG, deoC, ddpX, otsB, yhfZ, pheL, gmr, yibF, hdhA, ygdI, cysQ, yfdY, yceK, ydhC, yqjI, glpD, ydiZ, yhhA, yibT, ygjF, tam, yjfN, yacL, melR, yiaG, yjeF, ycfX, yiiT, kbl, dhaH, yafV, ycjX, ego, ytfR, sodC, ybaE, tdh, yhjD, fic, rpiB, ibpB, ytfN, yfiL, araB, relB, yadH, mrdB, ybiH, frwC, yjbR, psiF, basR, xylA, melA, ybhG, yhcO, garP, nlpE, ampE, ydaN, ydbC, pdxJ, glpT, fucA, yjbB, srlA, yddH, ybjK, yjgK, yadI, yncB, idi, sufA, zntR, ade, ynfD, yncG, yhcH, yhfY, yafM, yhhW, ybiI, yibI, ybeH, and ysaB.

In some aspects or embodiments, a genome-modified E. coli strain is provided that comprises at least 10 gene deletions or inactivations, including a plurality of gene deletions or inactivations selected from rpoS, hupB, clpB, uhpT, clpA, yeeI, htpG, motA, argT, rplP, flxA, fadD, pheT, flgM, arcA, modA, leuD, fumA, yjcZ, gpmA, rpoE, rseA, maeB, katG, ychH, hns, ygaT, cheW, ybhQ, atpC, glyS, fadB, osmE, hcaR, uspE, uspA, dps, ygeV, pheA, yfiQ, ibpA, ihfB, livK, miaA, ihfA, cysA, bolA, ydcS, ucpA, uspF, nuoH, yeaA, yjjY, aldB, yeaG, araC, yqfA, grpE, aroP, sra, ygjG, cheY, acrB, tpx, cysN, yaeH, osmC, ugpB, ykfB, Smg, yfcY, hypD, elaB, rsd, ybaL, fadH, ubiA, ygaU, sseA, yccV, bfr, pheS, yihX, ytfQ, putP, cycA, yeaT, yiiU, ycgB, rrmJ, rob, agp, ybeL, lrp, ydhR, malP, fbaB, msyB, glcC, ydcJ, ubiC, lon, glpK, dacC, yqjC, glmM, ompR, selD, ygdH, trpA, mukB, nupG, gltD, uspG, yggG, yrbL, glcD, cfa, phoH, yniA, gst, mscL, yhiO, ybhL, yahO, yjgD, glpF, wbbK, yfcX, yqjD, paaJ, yqjE, ynjH, aroL, galS, fruA, mhpR, rnr, prlC, smf, ygaM, ynhG, glgC, csiE, yaiZ, ygiN, aphA, ivy, glgS, ybjP, puuD, udp, yceP, fxsA, ybbN, yhbT, yeaY, ybeZ, ycjG, erfK, yhaO, yjdC, astC, sbmC, yghZ, ydbJ, uxuA, yhaL, fucO, ygdR, yajO, elbB, yhdW, ytfJ, ygfJ, ycfP, yfbT, phoU, blc, pepT, yqcA, yegP, yghU, sfsA, ysgA, yohC, mgtA, ybbK, nhaA, adhP, yehZ, rmuC, ydeW, yqeC, yecA, mppA, treA, dkgA, malS, ddlB, yafY, exuR, yhjY, yfbU, nadR, ydgD, qor, slt, yjgB, yaiE, soxS, prpR, yeiE, yahK, ydjF, yjfO, talA, yodD, pmrD, yhdH, yliB, yliJ, yeeZ, yaeP, ybdK, msrA, ydhQ, rfaB, ybjQ, ygiE, grxB, yjjM, ydhF, ycbB, araF, ygjR, yqjG, cdaR, ymgE, glnG, deoC, ddpX, otsB, yhfZ, pheL, gmr, yibF, hdhA, ygdI, cysQ, yfdY, yceK, ydhC, yqjI, glpD, ydiZ, yhhA, yibT, ygjF, tam, yjfN, yacL, melR, yiaG, yjeF, ycfX, yiiT, kbl, dhaH, yafV, ycjX, ego, ytfR, sodC, ybaE, tdh, yhjD, fic, rpiB, ibpB, ytfN, yfiL, araB, relB, yadH, mrdB, ybiH, frwC, yjbR, psiF, basR, xylA, melA, ybhG, yhcO, garP, nlpE, ampE, ydaN, ydbC, pdxJ, glpT, fucA, yjbB, srlA, yddH, ybjK, yjgK, yadI, yncB, idi, sufA, zntR, ade, ynfD, yncG, yhcH, yhfY, yafM, yhhW, ybiI, yibI, ybeH, and ysaB. In some embodiments, the E. coli strain further comprises a deletion or inactivation of one or more of (e.g., at least one, or at least two, or at least five of) fliC, aroF, aldA, cstA, aceA, cspD, aceB, trg, groL, dnaK, yfiA, gatC, flgL, flgK, acs, mdh, kgtP, fliA, glnH, and yjdA.

In still further embodiments, the bacterial strain has one or more genetic modifications to limit the stringent response. The stringent response is signaled by the alarmone (p)ppGpp. Such strains will produce less (p)ppGpp, which can otherwise impact growth and productivity under large scale conditions.

In various embodiments, the bacterial strain expresses a biosynthetic pathway (e.g., a recombinant biosynthetic pathway), to thereby produce a secondary metabolite product, including but not limited to a terpenoid, flavonoid, cannabinoid, polyketide, alkaloid, stilbenoid, polyphenol, amino acid, nucleotide, peptide, recombinant protein, or antibiotic. The bacterial strain may contain additional recombinant genes and/or genomic modifications to increase metabolic flux to the desired secondary metabolite. The modified genome strains described herein provide for improved energy and metabolic flux to the desired biosynthetic pathway, and avoids loss of productivity due to wasteful cellular processes.

In some aspects, the invention provides a method for making a product by large scale bioprocess. The method comprises culturing the strain described herein at large scale. In various embodiments, the strain is cultured in a bioreactor having a volume of at least about 1000 L, or at least about 10,000 L, or at least about 40,000 L, or at least about 100,000 L. In some embodiments, the bioreactor is a stirred tank bioreactor or a bubble column reactor. In some embodiments, the bacterial strain is used in a nutrient limited, fed batch fermentation process. In various embodiments, this disclosure provides for a prolonged production phase to thereby improve product yields by fed batch processes. In accordance with embodiments of the disclosure, the method can provide improvements in yield and/or purity of desired fermentation products.

Other aspects and embodiments of the invention will be apparent from the following detailed disclosure.

### DESCRIPTION OF FIGURES

FIG. 1 illustrates a STR-PFR (Stirred Tank Reactor-Plug Flow Reactor) for simulating large scale conditions.

FIG. 2 is a spider plot of E. coli genes grouped according to COG categories. The dotted line indicates the basic transcriptional level of all genes measured in a steady-state chemostat with installed dilution rate of 0.2 l/h not linked with the plug flow reactor. All gene transcript levels were set as reference.

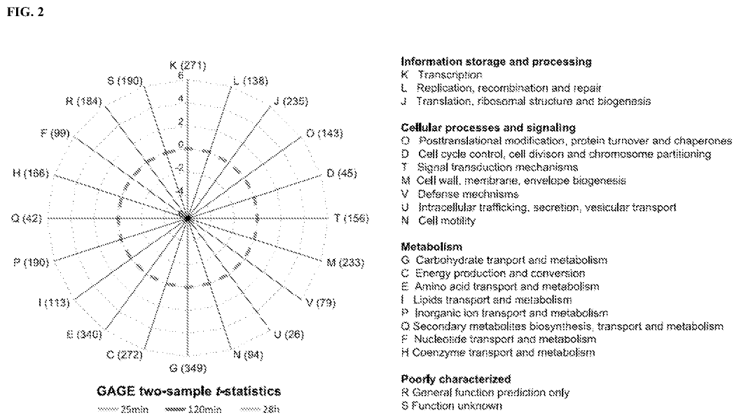

FIG. 3 is a spider plot of E. coli genes grouped according to COG categories. Lines indicate differentially expressed gene levels of one COG category measured after 25 min, 120 min, and 28 h of repeated exposure to glucose limitation inside PFR. Samples were taken at the outlet of PFR. The STR-PFR set was running with dilution rate of 0.2 l/h.

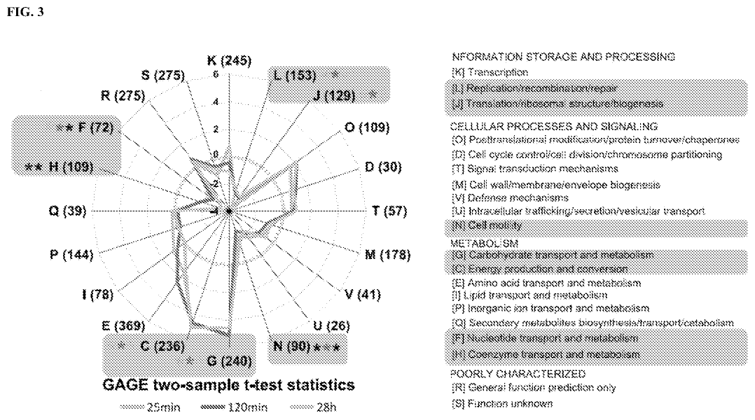

FIG. 4 provides a list of deletion strains (of E. coli MG1655) and their genotypes.

FIG. 5 shows that the deletion strains do not exhibit an impaired growth phenotype.

FIG. 6 shows that strain RM214 had a significantly lower maintenance coefficient than E. coli MG1655

FIG. 7 shows that E. coli RM214 remained significantly more productive than E. coli MG1655 after 28 h.

FIG. 8 shows that RM214 showed a significantly larger fraction of eGFP producers at 25 and 28 hours as compared to MG1655.

FIG. 9 shows that E. coli SR produces substantially less ppGpp.

FIG. 10 shows that E. coli SR has a dampened short term stress response, as shown by numbers of differentially expressed genes (DEGs).

FIG. 11 shows 2-dimensional principal component analysis of total transcripts measured in the stirred tank reactor after connection with PFR. Inside PFR, nitrogen limitation was repeatedly imposed on E. coli Wildtype (WT) and on E. coli SR, a stringent response deficient strain.

FIG. 12 shows differentially expressed genes of E. coli WT and E. coli SR grouped in COG categories and measured after frequent exposure to nitrogen limitation inside PFR (Top). Assignment of said gene transcripts to sigma factors is also shown (Bottom).

### DETAILED DESCRIPTION

In engineering cellular factories to take advantage of biochemical pathways, removal of wasteful auxiliary systems and optimization of pathway flux are opportunities to improve the productivity of these systems for the production of useful chemicals derived from secondary metabolic pathways. The advent of modern biotechnological tools allow us to redesign cellular metabolism and regulation in an intelligent, data-driven manner for the development of next generation cellular factories. However, in a cellular factory, there is a global interdependence of all cellular components, making it challenging to identify and remove unnecessary systems that decrease the overall efficiency. Removing these systems can convert the cell into a lean system for economical production of the desired product. The primary characteristics of such a cell would be reduced maintenance demand and an increase in productivity while preserving other physiologically relevant parameters.

The critical factor for developing such a platform cell factory is identifying and removing the unwanted cellular activities in the context of a large scale production process. This disclosure provides cell engineering approaches and cultivation systems for mimicking large scale fermentation, and provides scale down fermentation methods and applied omics analysis (e.g., transcriptomics and metabolomics) to identify systems level modulation of cellular activities under large scale cultivation scenario. This systems level modelling and analyses identify several cell functionalities as candidates for removal to develop a lean “cell factory” with reduced operating cost for economical production of a desired chemical product.

The present invention in various aspects and embodiments provides engineered bacterial strains having a modified genome and methods comprising large scale culture of the bacterial strains. In various embodiments, the strain engineering yields a reduction in cellular responses to micro-environmental stimuli imposed in large scale bioreactors. In some embodiments, the strain engineering allocates sufficient carbon and ATP even under resting cell conditions. Further, under large-scale production processes, metabolic activity is controlled, maintaining the process within the technical window of aeration, cooling, and mixing. Further, in some embodiments, the strain has a dampened stringent response.

In large-scale fed-batch production processes bacterial cells are exposed to heterogeneous substrate availability caused by long mixing times. E. coli, a common industrial host cell, when moving transiently through nutrient poor zones, reacts by looping accumulation of the alarmone ppGpp and energetically wasteful transcriptional strategies, resulting in growth and productivity limitations. In various aspects and embodiments of this disclosure, these limitations are ameliorated.

In one aspect, the invention provides a genome-modified bacterial strain. The bacterial strain comprises deletions or inactivations of non-essential genes, such as at least about 10 or at least about 20, or at least about 30 genes. Without being bound by theory, it is believed that the modifications avoid wasteful transcriptional or other cellular processes when the bacterial strain is cultured under large scale conditions. For example, the modifications reduce unnecessary requirements for DNA, RNA, and/or protein synthesis, or other energy-intensive cellular processes that require considerable ATP expenditure. Such gene deletions or inactivations include those coding for one, two, or more of: flagella components or transcriptional regulators thereof, chemotaxis proteins and regulators thereof, inhibitors of DNA replication, and proteins involved in active transport of sugars other than glucose. According to this aspect, the strain exhibits substantially normal growth and improved biosynthesis performance under large-scale conditions as compared to a parent strain that does not contain said deletions or inactivations.

Various bacterial strains may be employed according to this disclosure, including species of Escherichia, Bacillus, Corynebacterium, Pseudomonas, Rhodobacter, Zymomonas, Lactococcus, and Streptomyces, for example. Exemplary bacterial strains include strains of Escherichia coli, Bacillus subtilis, Corynebacterium glutamicum, Pseudomonas putida, Pseudomonas chlororaphis, Rhodobacter capsulatus, Rhodobacter sphaeroides, Zymomonas mobilis, Lactococcus lactis, and Streptomyces coelicolor. In various embodiments, the bacterial strain is a strain of Escherichia coli.

It is an object of the invention to provide bacterial strains whose growth and/or biosynthetic processes are not negatively impacted by large-scale conditions. In various embodiments, the large-scale conditions are at least about 1000 L, or at least about 10,000 L, or at least about 40,000 L, or at least about 100,000 L. As used herein, the term “about” means ±10% of an associated numerical value. In various embodiments, these objectives are realized by providing a bacterial strain having a modified genome. In general, the modified genome reduces requirements for DNA, RNA, and protein synthesis. For example, such strains may have deletions or transcriptional inactivations of at least about 15 genes, or at least about 20 genes, at least about 25 genes, at least about 30 genes, at least about 35 genes, or at least about 40 genes, or at least about 45 genes, or at least about 50 genes, or at least about 55 genes, or at least about 60 genes. In various embodiments, the genome size or transcriptional burden of (e.g., amount of RNA produced by) the bacterial strain with respect to native genes is reduced by at least about 1% from wild type, or at least about 2%, or at least about 3%, or at least about 4%, or at least about 5% from wild type. In various embodiments, the strain has a deletion or transcriptional inactivation of at least about 5 genes, or at least about 10 genes, or at least about 15 genes, or at least about 20 genes, or at least about 25 genes, or at least about 30 genes, or at least about 35 genes, or at least about genes coding for two or more of: flagella components or transcriptional regulators thereof, chemotaxis proteins and regulators thereof, inhibitors of DNA replication, and proteins involved in active transport of sugars other than glucose. In some embodiments, one or more transcription factors controlling key transcriptional units (e.g., one or more operons) is inactivated.

As used herein, the term “deletion” with respect to a gene means that the protein coding sequence of the gene is substantially or entirely deleted. In such embodiments, transcriptional regulatory sequences of the gene are optionally deleted, but may be retained in certain embodiments to retain transcription factor DNA binding sites and chromosome organization. Given that gene expression is inherently stochastic, changes in the balance of transcription factor availability and number of genome-binding sites may cause off-target effects in some cases. As used herein, the term “inactivation” includes transcriptional or translational inactivation. “Transcriptional inactivation” means that functional RNA transcripts of the gene are not produced or are substantially eliminated. Transcriptional inactivation can be conducted by deleting or substantially inactivating promoters or other cis-acting or trans-acting factors, such as by deleting or inactivating relevant transcription factors. Transcriptional inactivation can take place at the level of operons, in some embodiments. In still some embodiments, genes can be “translationally inactivated” such that protein synthesis from a transcript is abolished or substantially reduced. Translational inactivation can be conducted by modification or inactivation of ribosome binding sites or by introducing a premature stop codon. As used herein, the term “substantially reduced” with respect to transcriptional or translational inactivation requires at least a 50% decrease in the RNA or protein synthesis, depending on the context. In various embodiments, transcriptional or translational inactivation involves a decrease of at least about 75% or at least about 90% of RNA or protein synthesis (by mass) of the respective gene.

In various embodiments, the bacterial strain has deletions or inactivations of at least genes, or at least about 10 genes encoding flagella components or transcriptional regulators thereof. In some embodiments, the bacterial strain has deletions or inactivations of at least about 15 genes, or at least about 20 genes, or at least about 25 genes, or at least about 30 genes, encoding flagella components or transcriptional regulators thereof. The transcriptional organization of flagella genes is described in Fitzgerald D M, et al., Comprehensive Mapping of the Escherichia coli Flagellar Regulatory Network, PLOS Genetics 10:10 (2014). For example, in some embodiments, the bacterial strain is a strain of E. coli, and deleted or inactivated genes encoding flagella components or transcriptional regulators thereof are selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, and flhB. In embodiments that employ other bacterial species, flagella genes may be similarly deleted or inactivated, including orthologs of the E. coli genes listed in this paragraph.

In some embodiments, the bacterial strain contains deletions or transcriptional inactivations of full operons. In some embodiments, full operons or pathways are deleted, optionally along with corresponding regulatory transcription factors. For example, in some embodiments, the genes of the fliFGHIJK operon are deleted or transcriptionally inactivated and/or the genes of the fliMNOPQR operon are deleted or transcriptionally inactivated. In still other embodiments, one or more of fliA, fliC, and/or fliE are deleted, transcriptionally inactivated, or translationally inactivated. In some embodiments, flk is deleted, transcriptionally inactivated, or translationally inactivated. In some such embodiments, the genes of the flgBCDEGHIJ and/or flgKL operons are deleted or are transcriptionally inactivated. In some embodiments genes of the flhBAE operon are deleted or are transcriptionally inactivated.

In an exemplary embodiment, the E. coli strain comprises deletions or inactivations of fliA, flk, fliC, flgABCDEGHIJKL, fliEFGHIJKLMNOPQR, and flhEAB. In some embodiments, the strain has (or further comprises) a deletion or inactivation (transcriptional or translational) of one or both of motB and motA.

In these or other embodiments, expression of one or more flagella components can be prevented by translational inactivation or by protein mutation, thereby avoiding functional flagella assembly and thus avoid loss of ATP via active cell motility.

In these or other embodiments, the strain comprises deletions or inactivations of one or more genes encoding chemotaxis proteins and regulators thereof. For example, in some embodiments, the strain comprises a deletion or inactivation (transcriptional or translational) of fliA. Alternatively or in addition, the strain comprises deletions or inactivations of one or more of E. coli genes tar, tap, cheR, cheB, cheY, cheZ, cheW, and cheA (or orthologs thereof with respect to other bacterial species). For example, in some embodiments, the strain comprises a deletion or transcriptional inactivation of genes of the operon tar-tap-cheRBYZ, and/or the operon motAB-cheAW.

In various embodiments, the strain comprises a deletion or inactivation (transcriptional or translational) of at least one gene encoding an inhibitor of DNA replication. An exemplary deletion is cspD, or an ortholog thereof.

In some embodiments, the strain has a deletion or inactivation of one or more genes involved in active transport of sugars, especially sugars other than glucose. For example, deletion or inactivation of sugar transport pathways that are not needed based on the carbon substrates employed during culture can be helpful to reduce wasteful cellular processes. Sugar transport in bacteria involves membrane proteins that catalyze translocation and accumulation of various sugars from the outside environment. In bacteria, there are three types of systems involved in sugar transport, and each one utilizes distinct energy sources. In some embodiments, deleted or inactivated genes are phosphotransferase system (PTS) proteins, including in some embodiments, membrane transporters. In some embodiments, genes involved in galactitol transport or metabolism are deleted or inactivated. Exemplary genes according to these embodiments include one or more of E. coli gatA, gatB, gatC, gatD, gatR, and uhpT (or orthologs thereof). In some embodiments, the strain comprises a deletion or transcriptional inactivation of gatABCDR as well deletion or inactivation of uhpT.

In still other embodiments, the bacterial strain comprises deletions or inactivations of other non-essential genes, such as one or more of E. coli aldA, yeeI, yeeL, and flxA (or orthologs thereof), as well as others described herein.

Thus, in some embodiments, the bacterial strain comprises at least 10 deletions or inactivations, or at least about 20 deletions or inactivations, or at least about 30 deletions or inactivation (or all deletions or inactivations) selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

In exemplary strains in accordance with embodiments described above, the bacterial strain is a strain of E. coli comprising deletions or inactivations selected from fliC, aroF, aldA, cstA, aceA, cspD, aceB, trg, groL, dnaK, yfiA, gatC, flgL, flgK, acs, mdh, kgtP, fliA, glnH, yjdA, rpoS, hupB, clpB, uhpT, clpA, yeeI, htpG, motA, argT, rplP, flxA, fadD, pheT, flgM, arcA, modA, leuD, fumA, yjcZ, gpmA, rpoE, rseA, maeB, katG, ychH, hns, ygaT, cheW, ybhQ, atpC, glyS, fadB, osmE, hcaR, uspE, uspA, dps, ygeV, pheA, yfiQ, ibpA, ihfB, livK, miaA, ihfA, cysA, bolA, ydcS, ucpA, uspF, nuoH, yeaA, yjjY, aldB, yeaG, araC, yqfA, grpE, aroP, sra, ygjG, cheY, acrB, tpx, cysN, yaeH, osmC, ugpB, ykfB, Smg, yfcY, hypD, elaB, rsd, ybaL, fadH, ubiA, ygaU, sseA, yccV, bfr, pheS, yihX, ytfQ, putP, cycA, yeaT, yiiU, ycgB, rrmJ, rob, agp, ybeL, lrp, ydhR, malP, fbaB, msyB, glcC, ydcJ, ubiC, lon, glpK, dacC, yqjC, glmM, ompR, selD, ygdH, trpA, mukB, nupG, gltD, uspG, yggG, yrbL, glcD, cfa, phoH, yniA, gst, mscL, yhiO, ybhL, yahO, yjgD, glpF, wbbK, yfcX, yqjD, paaJ, yqjE, ynjH, aroL, galS, fruA, mhpR, rnr, prlC, smf, ygaM, ynhG, glgC, csiE, yaiZ, ygiN, aphA, ivy, glgS, ybjP, puuD, udp, yceP, fxsA, ybbN, yhbT, yeaY, ybeZ, ycjG, erfK, yhaO, yjdC, astC, sbmC, yghZ, ydbJ, uxuA, yhaL, fucO, ygdR, yajO, elbB, yhdW, ytfJ, ygfJ, ycfP, yfbT, phoU, blc, pepT, yqcA, yegP, yghU, sfsA, ysgA, yohC, mgtA, ybbK, nhaA, adhP, yehZ, rmuC, ydeW, yqeC, yecA, mppA, treA, dkgA, malS, ddlB, yafY, exuR, yhjY, yfbU, nadR, ydgD, qor, slt, yjgB, yaiE, soxS, prpR, yeiE, yahK, ydjF, yjfO, talA, yodD, pmrD, yhdH, yliB, yliJ, yeeZ, yaeP, ybdK, msrA, ydhQ, rfaB, ybjQ, ygiE, grxB, yjjM, ydhF, ycbB, araF, ygjR, yqjG, cdaR, ymgE, glnG, deoC, ddpX, otsB, yhfZ, pheL, gmr, yibF, hdhA, ygdI, cysQ, yfdY, yceK, ydhC, yqjI, glpD, ydiZ, yhhA, yibT, ygjF, tam, yjfN, yacL, melR, yiaG, yjeF, ycfX, yiiT, kbl, dhaH, yafV, ycjX, ego, ytfR, sodC, ybaE, tdh, yhjD, fic, rpiB, ibpB, ytfN, yfiL, araB, relB, yadH, mrdB, ybiH, frwC, yjbR, psiF, basR, xylA, melA, ybhG, yhcO, garP, nlpE, ampE, ydaN, ydbC, pdxJ, glpT, fucA, yjbB, srlA, yddH, ybjK, yjgK, yadI, yncB, idi, sufA, zntR, ade, ynfD, yncG, yhcH, yhfY, yafM, yhhW, ybiI, yibI, ybeH, and ysaB.

In some aspects or embodiments (including according to embodiments described above), a genome-modified E. coli strain is provided that comprises at least 10 gene deletions or inactivations (or at least 20 gene deletions or inactivations, or at least 25 gene deletions or inactivations, or at least 30 gene deletions or inactivations), including a plurality of gene deletions or inactivations (e.g., at least 5, at least 10, at least 15, at least 20, at least 25, or at least 30 genes) selected from, rpoS, hupB, clpB, uhpT, clpA, yeeI, htpG, motA, argT, rplP, flxA, fadD, pheT, flgM, arcA, modA, leuD, fumA, yjcZ, gpmA, rpoE, rseA, maeB, katG, ychH, hns, ygaT, cheW, ybhQ, atpC, glyS, fadB, osmE, hcaR, uspE, uspA, dps, ygeV, pheA, yfiQ, ibpA, ihfB, livK, miaA, ihfA, cysA, bolA, ydcS, ucpA, uspF, nuoH, yeaA, yjjY, aldB, yeaG, araC, yqfA, grpE, aroP, sra, ygjG, cheY, acrB, tpx, cysN, yaeH, osmC, ugpB, ykfB, Smg, yfcY, hypD, elaB, rsd, ybaL, fadH, ubiA, ygaU, sseA, yccV, bfr, pheS, yihX, ytfQ, putP, cycA, yeaT, yiiU, ycgB, rrmJ, rob, agp, ybeL, lrp, ydhR, malP, fbaB, msyB, glcC, ydcJ, ubiC, lon, glpK, dacC, yqjC, glmM, ompR, selD, ygdH, trpA, mukB, nupG, gltD, uspG, yggG, yrbL, glcD, cfa, phoH, yniA, gst, mscL, yhiO, ybhL, yahO, yjgD, glpF, wbbK, yfcX, yqjD, paaJ, yqjE, ynjH, aroL, galS, fruA, mhpR, rnr, prlC, smf, ygaM, ynhG, glgC, csiE, yaiZ, ygiN, aphA, ivy, glgS, ybjP, puuD, udp, yceP, fxsA, ybbN, yhbT, yeaY, ybeZ, ycjG, erfK, yhaO, yjdC, astC, sbmC, yghZ, ydbJ, uxuA, yhaL, fucO, ygdR, yajO, elbB, yhdW, ytfJ, ygfJ, ycfP, yfbT, phoU, blc, pepT, yqcA, yegP, yghU, sfsA, ysgA, yohC, mgtA, ybbK, nhaA, adhP, yehZ, rmuC, ydeW, yqeC, yecA, mppA, treA, dkgA, malS, ddlB, yafY, exuR, yhjY, yfbU, nadR, ydgD, qor, slt, yjgB, yaiE, soxS, prpR, yeiE, yahK, ydjF, yjfO, talA, yodD, pmrD, yhdH, yliB, yliJ, yeeZ, yaeP, ybdK, msrA, ydhQ, rfaB, ybjQ, ygiE, grxB, yjjM, ydhF, ycbB, araF, ygjR, yqjG, cdaR, ymgE, glnG, deoC, ddpX, otsB, yhfZ, pheL, gmr, yibF, hdhA, ygdI, cysQ, yfdY, yceK, ydhC, yqjI, glpD, ydiZ, yhhA, yibT, ygjF, tam, yjfN, yacL, melR, yiaG, yjeF, ycfX, yiiT, kbl, dhaH, yafV, ycjX, ego, ytfR, sodC, ybaE, tdh, yhjD, fic, rpiB, ibpB, ytfN, yfiL, araB, relB, yadH, mrdB, ybiH, frwC, yjbR, psiF, basR, xylA, melA, ybhG, yhcO, garP, nlpE, ampE, ydaN, ydbC, pdxJ, glpT, fucA, yjbB, srlA, yddH, ybjK, yjgK, yadI, yncB, idi, sufA, zntR, ade, ynfD, yncG, yhcH, yhfY, yafM, yhhW, ybiI, yibI, ybeH, and ysaB. In some embodiments, the E. coli strain further comprises a deletion or inactivation of one or more of (e.g., at least one, or at least two, or at least five of) fliC, aroF, aldA, cstA, aceA, cspD, aceB, trg, groL, dnaK, yfiA, gatC, flgL, flgK, acs, mdh, kgtP, fliA, glnH, and yjdA. In some embodiments, the genome modifications are solely via gene deletion. In some embodiments, the genome modifications are predominately deletions and transcriptional inactivations.

In still further embodiments, the bacterial strain has one or more genetic modifications to limit the stringent response. The stringent response is signaled by the alarmone (p)ppGpp. Such strains will produce less (p)ppGpp, which can otherwise impact growth and productivity under large scale conditions. In various embodiments, the bacterial strain comprises a deletion or inactivation of relA, and/or comprises a spoT mutant (e.g., a loss of function mutation). In some embodiments, the spoT mutant comprises the amino acid substitutions R290E and K292D (with respect to the wild type E. coli protein). For example, the combination of ΔrelA, spoT mutation, and mutated pyruvate dehydrogenase AceE in E. coli enable alleviated glucose uptake for resting cells, but may exhibit reduced growth rates. Consequently, co-integration of heterologous glucose facilitator glf of Zymomonas mobilis (SEQ ID NO: 1) and cytosolic glucokinase (glk) (e.g, Zymomonas mobilis, SEQ ID NO: 2) (or orthologs thereof) can be implemented. Thus, in some embodiments, the bacterial strain further comprise an aceE mutant, which optionally comprises a G267C substitution in aceE (with respect to wild type). Thus, in some embodiments, the bacterial strain comprises an overexpression or complementation of a gene that increases glucose uptake. For example, the bacterial strain comprises recombinant expression of one or more of SEQ ID NO:1 and/or SEQ ID NO: 2, or derivatives thereof. Derivatives of SEQ ID NO: 1 and SEQ ID NO: 2 may comprise amino acid sequences having at least about 70%, or at least about 75%, or at least about 80%, or at least about 85%, or at least about 90%, or at least about 95%, or at least about 97%, or at least about 98% sequence identity with SEQ ID NO: 1 or SEQ ID NO: 2. In some embodiments, one or more phosphotransferase system (PTS) proteins involved in glucose transport are overexpressed.

In various embodiments, the bacterial strain provides advantages in maintenance. In some embodiments, the strain has a maintenance coefficient of less than 0.14 g_{glucose}/g_{CDW}/h at large scale, or about 0.12 g_{glucose}/g_{CDW}/h or less, or about 0.10 g_{glucose}/g_{CDW}/h or less at large scale.

In various embodiments, the bacterial strain expresses a biosynthetic pathway (e.g., a recombinant biosynthetic pathway), to thereby produce a secondary metabolite product, including but not limited to a terpenoid, flavonoid, cannabinoid, polyketide, alkaloid, stilbenoid, and polyphenol. The bacterial strain may contain additional recombinant genes (including genes for a plant biosynthetic pathway) and other supporting genes or genomic modifications. Other products of interest include amino acids, nucleotides, fatty acids, antibiotics, recombinant proteins, and peptides. The bacterial strain may contain additional recombinant genes and/or genomic modifications to increase metabolic flux to the desired secondary metabolite or product. For example, the modified genome strains described herein provide for improved available energy and metabolic flux to the desired biosynthetic pathway, and avoids loss of productivity due to wasteful cellular processes.

In certain embodiments, the bacterial strain produces one or more terpene or terpenoid compounds. In an embodiment, the bacterial strain produces a terpenoid selected from a monoterpenoid, a sesquiterpenoid, diterpenoid, a sesterpenoid, or a triterpenoid. Terpenoids represent a diverse class of molecules that provide numerous commercial applications, including in the food and beverage industries as well as the perfume, cosmetic and health care industries. By way of example, terpenoid compounds find use in perfumery (e.g. patchoulol, rotundone), in the flavor industry (e.g., nootkatone), as sweeteners (e.g., steviol glycoside or mogroside), colorants, or therapeutic agents (e.g., taxol or artemisinin) and many are conventionally extracted from plants. Nevertheless, terpenoid molecules are found in ppm levels in nature, and therefore require massive harvesting to obtain sufficient amounts for commercial applications.

For example, the bacterial strain may comprise a recombinant downstream pathway that produces the terpenoid from IPP and DMAPP precursors. Terpenes such as Monoterpenes (C10), Sesquiterpenes (C15), Diterpenes (C20), Sesterterpenes (C25), and Triterpenes (C30) are derived from the prenyl diphosphate substrates, geranyl diphosphate (GPP), farnesyl diphosphate (FPP) geranylgeranyl diphosphate (GGPP), geranylfarnesyl diphosphate (FGPP), and two FPP, respectively, through the action of a very large group of enzymes called the terpene (terpenoid) synthases. These enzymes are often referred to as terpene cyclases since the product of the reactions are cyclized to various monoterpene, sesquiterpene, diterpene, sesterterpene and triterpene carbon skeleton products. Many of the resulting carbon skeletons undergo subsequence oxygenation by cytochrome P450 enzymes to give rise to large families of derivatives.

Exemplary terpene or terpenoid products that may be produced are described in U.S. Pat. No. 8,927,241, which is hereby incorporated by reference, and include: farnesene, amorphadiene, artemisinic acid, artemisinin, bisabolol, bisabolene, alpha-Sinensal, beta-thuj one, camphor, carveol, carvone, cineole, citral, citronellal, cubebol, geraniol, limonene, menthol, menthone, mogrol or mogrol glycoside, myrcene, nootkatone, nootkatol, patchouli, piperitone, rose oxide, rotundol, rotundone, sabinene, steviol, steviol glycoside (including Rebaudioside D or Rebaudioside M), taxadiene, thymol, and valencene. Enzymes for recombinantly constructing the pathways in E. coli are described in U.S. Pat. Nos. 8,927,241, 10,463,062, and U.S. Patent Application Publication No. 2018/0135081, and published PCT Application WO 2019/169027, which are hereby incorporated by reference.

In some embodiments, the strain has an overexpression of one or more MEP pathway genes. In some embodiments, the strain expresses a terpenoid biosynthesis pathway. In some embodiments, the microbial strain has at least one additional copy of dxs, ispD, ispF, and/or idi genes, which can be rate limiting with respect to the MEP pathway, and which can be expressed from an operon or module, either on a plasmid or integrated into the bacterial chromosome. In some embodiments, the bacterial strain has at least one additional copy of dxs and idi expressed as an operon/module; or dxs, ispD, ispF, and idi expressed as an operon or module. In some embodiments, the bacterial strain expresses dxs, dxr, ispD, ispE, ispF, and idi as recombinant genes, which are optionally expressed as 1, 2, or 3 individual operons or modules. The recombinant genes of the MEP pathway are expressed from one or more plasmids or are integrated into the chromosome. In these embodiments, the strain provides increased flux through the MEP pathway as compared to wild type.

In some embodiments, the bacterial strain is engineered to increase production of IPP and DMAPP from glucose as described in U.S. Pat. Nos. 10,480,015 and 10,662,442, the contents of which are hereby incorporated by reference in their entireties. For example, in some embodiments the host cell overexpresses MEP pathway enzymes, with balanced expression to push/pull carbon flux to IPP and DMAPP.

For example, in some embodiments, the bacterial strain is engineered to increase the availability or activity of Fe—S cluster proteins, so as to support higher activity of IspG and IspH, which are Fe—S enzymes. For example, the bacterial strain may contain one or more genetic modifications that enhance the supply and transfer of electrons through the MEP pathway, and/or to terpene or terpenoid products. In some embodiments, the enhanced supply and transfer of electrons through the MEP pathway is by recombinant expression of one or more oxidoreductase enzymes, including oxidoreductases that oxidize pyruvate and/or lead to reduction of ferredoxin. Ferredoxin supplies electrons to the MEP pathway and supports activity of IspG and IspH. In various embodiments, the bacterial strain comprises an overexpression of or complementation with one or more of a flavodoxin (fldA), flavodoxin reductase, ferredoxin (fdx), and ferredoxin reductase.

By way of example, the oxidoreductase may be a pyruvate:flavodoxin oxidoreductase (PFOR). In some embodiments, the PFOR is E. coli Ydbk (SEQ ID NO: 3), or ortholog or derivative thereof. Derivative enzymes generally comprise amino acid sequences having at least about 70%, or at least about 80% or at least about 90%, or at least about 95% sequence identity with E. coli Ydbk (SEQ ID NO: 3). Additionally, extra electron-carrying or transferring cofactors can be expressed on top of YdbK overexpression. In some experiments, YdbK is overexpressed with fdc (ferredoxin) from Clostridium pasteurianum and/or E. coli ydhY, or enzyme having at least 80% or at least 90% sequence identity therewith.

In some embodiments, including in embodiments where the bacterial strain overexpresses or has higher activity of pyruvate:flavodoxin oxidoreductase (PFOR), the strain may exhibit reduced conversion of pyruvate to acetyl-COA by pyruvate dehydrogenase (PDH). In some embodiments, the conversion of pyruvate to acetyl-COA by PDH is reduced by deleting or inactivating PDH, or by reducing expression or activity of PDH. In some embodiments, PDH is deleted. Alternatively, activity of PDH may be reduced by one or more amino acid modifications. An exemplary mutation to reduce PDH activity is a G267C mutation in AceE.

In some embodiments, the conversion of pyruvate to acetyl-COA by PDH is reduced by modifying the aceE-aceF-lpd complex of PDH. In some embodiments, the aceE-aceF-lpd complex is modified by the deletion, inactivation, or reduced expression or activity of aceE, aceF, lpd, or a combination thereof. By way of example, in some embodiments, aceE is deleted (e.g., by knockout). Alternatively, in some embodiments, the aceE-aceF-lpd complex is modified by one or more mutations of aceE, aceF, lpd, or a combination thereof.

By reducing conversion of pyruvate to acetyl-COA by PDH, the bacterial strain will rely more on PFOR (e.g., YdbK) for the conversion of pyruvate to acetyl-COA. This reliance enhances IspG and IspH activity.

In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux and/or terpenoid production. In some embodiments, the host cell exhibits higher activity of IspH relative to IspG. For example, IspH and IspG may be expressed from an operon, where IspH is positioned first. In some embodiments, the host cell is engineered to downregulate the ubiquinone biosynthesis pathway, e.g., by reducing the expression or activity of IspB, which uses IPP and FPP substrate.

In some embodiments, as an alternative to MEP pathway engineering, the bacterial strain may express an Isopentenol Utilization Pathway (IUP), as described in US 2019/0367950, which is hereby incorporated by reference in its entirety. For example, the bacterial strain may express one or more recombinantly expressed enzymes that phosphorylate isoprenol and/or prenol (which is added as a substrate to the culture) to produce isopentenyl diphosphate (IPP) and/or dimethylallyl diphosphate (DMAPP). In some embodiments, the one or more recombinantly expressed enzymes comprises an amino acid sequence that has at least about 70% sequence identity to the amino acid sequence of SEQ ID NO: 4 and capable of catalyzing the synthesis of IPP and DMAPP from isoprenol and/or prenol. In some embodiments, the enzyme capable of catalyzing the synthesis of IPP and DMAPP from isoprenol and/or prenol comprises an amino acid sequence that has at least about 80% sequence identity, or at least about 85% sequence identity, or at least about 90% sequence identity, or at least about 95% sequence identity, or at least about 97% sequence identity to SEQ ID NO: 4. In some embodiments, the cell further expresses a recombinant isopentenyl phosphate kinase (e.g., from Arabidopsis thaliana or derivative thereof having at least about 70%, at least about 80%, or about 90%, or about 95%, or about 97% sequence identity thereto), and an overexpression of (e.g., gene complementation) isopentenyl pyrophosphate isomerase (IDI).

Other biosynthetic pathways that can be employed include those for the production of flavonoids, as described in U.S. Pat. No. 9,181,539 (which is hereby incorporated by reference in its entirety), or cannabinoids, as described in WO 2020/102541 or US 2019/0382813 (which are hereby incorporated by reference in its entirety), among others.

In some embodiments, the strain expresses at least one recombinant cytochrome P450 enzyme, which may be a plant P450 enzyme. The P450 enzyme provides for oxidative chemistry on the molecular scaffold of interest, which may be produced by the host cell or fed to the culture. In some embodiments, the cytochrome P450 enzyme has at least a portion of its transmembrane region substituted with a heterologous transmembrane region. For example, the CYP450 and/or reductase partner is modified as described in U.S. Pat. No. 10,774,314, the contents of which are hereby incorporated by reference in their entireties. For example, in some embodiments, the CYP450 enzyme has a deletion of all or part of the wild type P450 N-terminal transmembrane region, and the addition of a transmembrane domain derived from an E. coli or bacterial inner membrane, cytoplasmic C-terminus protein. In some embodiments, the transmembrane domain is a single-pass transmembrane domain. In some embodiments, the transmembrane domain is a multi-pass (e.g., 2, 3, or more transmembrane helices) transmembrane domain. Exemplary E. coli inner membrane proteins include waaA, ypfN, yhcB, yhbM, yhhm, zipA, ycgG, djlA, sohB, IpxK, F110, motA, htpx, pgaC, ygdD, hemr, and ycls.

In some embodiments, the strain expresses at least one uridine diphosphate-dependent glycosyl transferase (UGT) enzyme, which may be used in a whole cell bioconversion process. Such strains have higher dependence on glucose. Whole cell bioconversion processes using bacterial cells (including E. coli) are described in US 2020/0087692, which is hereby incorporated by reference in its entirety. The bacterial cell may be engineered to increase availability of UDP-glucose. For example, the bacterial cell may have a deletion, inactivation, or reduced activity or expression of a gene encoding an enzyme that consumes UDP-glucose. For example, the bacterial cell may have a deletion, inactivation, or reduced activity of ushA (UDP-sugar hydrolase) and/or one or more of galE, galT, galK, and galM (which are responsible for UDP-galactose biosynthesis from UDP-glucose), or ortholog thereof in the bacterial species. In some embodiments, galETKM genes are inactivated, deleted, or substantially reduced in expression. Alternatively or in addition, the bacterial cell has a deletion, inactivation, or reduced activity or expression of E. coli otsA (trehalose-6-phosphate synthase), or ortholog thereof in the bacterial species. Alternatively or in addition, the bacterial cell has a deletion, inactivation, or reduced activity or expression of E. coli ugd (UDP-glucose 6-dehydrogenase), or ortholog thereof in the bacterial species. Reducing or eliminating activity of otsA and ugd can remove or reduce UDP-glucose sinks to trehalose or UDP-glucuronidate, respectively. Other UDP-glucose sinks that can be reduced or eliminated include eliminating or reducing activity or expression of genes responsible for lipid glycosylation and LPS biosynthesis, and genes responsible for glycosylating undecaprenyl-diphosphate (UPP).

In these or other embodiments, the bacterial cell has a deletion, inactivation, or reduced activity or expression of a gene encoding an enzyme that consumes a precursor to UDP-glucose. For example, in some embodiments, the bacterial cell has a deletion, inactivation, or reduced activity or expression of pgi (glucose-6 phosphate isomerase), or ortholog thereof in the bacterial species of the host cell. In these or other embodiments, the bacterial cell has an overexpression or increased activity of one or more genes encoding an enzyme involved in converting glucose-6-phosphate to UDP-glucose. For example, pgm (phosphoglucomutase) and/or galU (UTP-glucose-1-phosphate uridylyltransferase) (or ortholog or derivative thereof) can be overexpressed, or modified to increase enzyme productivity. Alternatively or in addition, E. coli ycjU (β-phosphoglucomutase), which converts glucose-6-phosphate to glucose-1-phosphate, and Bifidobacterium bifidum ugpA, which converts glucose-1-phosphate to UDP, or ortholog or derivative of these enzymes, can be overexpressed, or modified to increase enzyme productivity.

Alternatively or in addition, the bacterial cell has one or more genetic modifications that increase glucose transport. Such modifications include increased expression or activity of E. coli galP (galactose:H+symporter) and E. coli glk (glucokinase), or alternatively Zymomonas mobilis glf and E. coli glk, or homologues, orthologs, or engineered derivatives of these genes.

Alternatively or in addition, the microbial cell has one or more genetic modifications that increase UTP production and recycling. Such modifications include increased expression or activity of E. coli pyrH (UMP kinase), E. coli cmk (cytidylate kinase), E. coli adk (adenylate kinase), or E. coli ndk (nucleoside diphosphate kinase), or homologs, orthologs, or engineered derivatives of these enzymes.

Alternatively or in addition, the microbial cell has one or more genetic modifications that increase UDP production. Such modifications include overexpression or increased activity of one or more of E. coli upp (uracil phosphoribosyltransferase), E. coli dctA (C4 dicarboxylate/orotate:H+symporter), E. coli pyrE (orotate phosphoribosyltransferase), and E. coli pyrF (orotidine-5′-phosphate decarboxylase), including homologs, orthologs, or engineered derivatives thereof. For example, in some embodiments, the microbial cell overexpresses or has increased activity of upp, pyrH and cmk, or homolog or engineered derivative thereof. Alternatively, the microbial cell overexpresses or has increased activity of dctA, pyre, pyrH and cmk, or homolog or engineered derivative thereof.

Alternatively or in addition, the microbial cell may have one or more genetic modifications to remove or reduce regulation of glucose uptake. For example, the microbial cell may have a deletion, inactivation, or reduced expression of sgrS, which is a small regulatory RNA in E. coli.

Alternatively or in addition, the microbial cell may have one or more genetic modifications that reduce dephosphorylation of glucose-1-phosphate. Exemplary modifications include deletion, inactivation, or reduced expression or activity of one or more of E. coli agp (glucose-1-phosphatase), E. coli yihX (.alpha.-D-glucose-1-phosphate phosphatase), E. coli ybiV (sugar phosphatase), E. coli yidA (sugar phosphatase), E. coli yigL (phosphosugar phosphatase), and E. coli phoA (alkaline phosphatase), or an ortholog thereof in the microbial cell.

Alternatively or in addition, the microbial cell may have one or more genetic modifications that reduce conversion of glucose-1-phosphate to TDP-glucose. Exemplary modifications include deletion, inactivation, or reduced expression or activity of one or more of E. coli rffH (dTDP-glucose pyrophosphorylase) and E. coli rfbA (dTDP glucose pyrophosphorylase), or an ortholog thereof in the microbial cell.

Alternatively or in addition, the microbial cell may have one or more genetic modifications that reduce conversion of glucose-1-phosphate to ADP-glucose. Exemplary modifications include deletion, inactivation, or reduced expression or activity of E. coli gigC (glucose-1-phosphate adenylyltransferase), or an ortholog thereof in the microbial cell.

In some embodiments, the microbial cell is a bacterial cell comprising the genetic modifications: ushA and galETKM are deleted, inactivated, or reduced in expression; pgi is deleted, inactivated, or reduced in expression; and pgm and galU are overexpressed or complemented.

Amino acid modifications can be made to enzymes to increase or decrease activity of the enzyme or enzyme complex. Gene mutations can be performed using any genetic mutation method known in the art. In some embodiment, a gene knockout eliminates a gene product in whole or in part. Gene knockouts can be performed using any knockout method known in the art. Manipulation of the expression of genes and/or proteins, including gene modules, can be achieved through various methods. For example, expression of the genes or operons can be regulated through selection of promoters, such as inducible or constitutive promoters, with different strengths (e.g., strong, intermediate, or weak). Several non-limiting examples of promoters include Trc, T5 and T7. Additionally, expression of genes or operons can be regulated through manipulation of the copy number of the gene or operon in the cell. In some embodiments, expression of genes or operons can be regulated through manipulating the order of the genes within a module, where the genes transcribed first are generally expressed at a higher level. In some embodiments, expression of genes or operons is regulated through integration of one or more genes or operons into the chromosome. In some embodiments, expression of genes are modulated by modification of the ribosome binding sequence, as described in U.S. Pat. No. 10,662,442, which is hereby incorporated by reference.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Bacterial cells are genetically engineered by the introduction into the cells of heterologous DNA. The heterologous DNA is placed under operable control of transcriptional elements to permit the expression of the heterologous DNA in the host cell. In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

In some embodiments, the strain produces one or more secondary metabolites of interest, without recombinant expression of a biosynthetic pathway. For example, in some embodiments, the secondary metabolite of interest is an antibiotic (e.g., using Streptomyces spp. or other antibiotic producing species), an amino acid (including with reduced genome strains of Corynebacterium glutamicum in accordance with this disclosure), or a nucleotide product. Exemplary antibiotics that may be produced include Chloramphenicol, Lincomycin, Neomycin, and Tetracycline.

In still other embodiments, the strain produces a recombinant protein product, such as an industrial enzyme, plant protein or enzyme, or therapeutic protein. In such embodiments, the bacterial strain may be a strain of E. coli or Bacillus subtilis, for example.

In some aspects, the invention provides a method for making a product by large scale fermentation. The method comprises culturing the strain described herein at large scale. In various embodiments, the strain is cultured in a bioreactor having a volume of at least about 1000 L, or at least about 10,000 L, or at least about 40,000 L, or at least about 100,000 L. In some embodiments, the bioreactor is a stirred tank bioreactor. In some embodiments, the bioreactor is a bubble column reactor.

The bacterial strain may be cultured in batch culture, continuous culture, or semi-continuous culture. In some embodiments, the bacterial strain is cultured using a fed-batch process comprising a first phase where bacterial biomass is created, followed by a production phase. Fed-batch culture is a process where nutrients are fed to the bioreactor during cultivation and in which the product(s) remain in the bioreactor until the end of the run. Generally, a base medium supports initial cell culture and a feed medium is added to prevent nutrient depletion. The controlled addition of the nutrient directly affects the growth rate of the culture and helps to avoid overflow metabolism and formation of side metabolites.

In some embodiments, the bacterial strain is used in a nutrient limited (e.g., glucose limited) fed batch fermentation process. In such embodiments, the bacterial strain provides improvements in biomass production and/or improvements in the production phase. In some embodiments, the bacterial strain is used with an ammonium limited fed-batch process, for example, with a prolonged nitrogen-limited production phase. In various embodiments, this disclosure provides for a prolonged production phase of about 24 hours or more, or about 36 hours or more, or about 48 hours or more, or about 60 hours or more, or about 72 hours or more.

An exemplary batch media for growing the bacterial strain (producing biomass) comprises, without limitation, yeast extract. In some embodiments, carbon substrates such C1, C2, C3, C4, C5, and/or C6 carbon substrates are fed to the culture for production of the desired product. In exemplary embodiments, the carbon source is, or comprises, glucose, sucrose, fructose, xylose, and/or glycerol. Culture conditions are generally selected from aerobic, microaerobic, and anaerobic.

In some embodiments, the culture is maintained under aerobic conditions, or microaerobic conditions. For example, when using a fed-batch process, the biomass production phase can take place under aerobic conditions, followed by reducing the oxygen levels for the product production phase. For example, the culture can be shifted to microaerobic conditions after from about 10 to about 20 hours. In this context, the term “microaerobic conditions” means that cultures are maintained just below detectable dissolved oxygen. See, Partridge J D, et al., Transition of Escherichia coli from Aerobic to Micro-aerobic Conditions Involves Fast and Slow Reacting Regulatory Components, J. Biol. Chem. 282(15):11230-11237 (2007).

The production phase includes feeding a nitrogen source and a carbon source. For example, the nitrogen source can comprise ammonium (e.g., ammonium hydroxide). The carbon source may comprise, in some embodiments, glucose, sucrose, or glycerol. The nitrogen and carbon feeding can be initiated when a predetermined amount of batch media is consumed, a process that provides for ease of scaling. In some embodiments, the nitrogen feed rate is from about 8 L per hour to about 20 L per hour, but will depend in-part on the product, strain, and scale.

In various embodiments, the bacterial host cell may be cultured at a temperature from about 22° C. to about 37° C. While commercial biosynthesis in bacteria such as E. coli can be limited by the temperature at which overexpressed and/or foreign enzymes are stable, recombinant enzymes may be engineered to allow for cultures to be maintained at higher temperatures, resulting in higher yields and higher overall productivity. In some embodiments, the culturing is conducted at about 25° C. or greater, about 30° C. or greater, about 32° C. or greater, or about 34° C. or greater, or about 37° C. in some embodiments. In some embodiments, the culture is maintained at a temperature of from about 25 to about 37° C., or a temperature of from about 27 to about 37° C., or a temperature of from about 30 to about 37° C.

In various embodiments, methods further include recovering the product from the cell culture or from cell lysates. In various embodiments, the amount of product produced and recovered will depend on, for example, the type of product being produced, the level of strain or enzyme engineering, and the culture conditions. In various embodiments, the culture produces at least about 100 mg/L, at least about 150 mg/L, or at least about 200 mg/L, or at least about 500 mg/L, or at least about 1 g/L, or at least about 5 g/L, or at least about 10 g/L, or at least about 15 g/L of the product, or at least about 25 g/L of the product, or at least about 50 g/L of the product.

In some embodiments, for the production of terpenoids, the production of indole is used as a surrogate marker for terpenoid production, and/or the accumulation of indole in the culture is controlled to increase production. For example, in various embodiments, accumulation of indole in the culture is controlled to below about 100 mg/L, or below about mg/L, or below about 50 mg/L, or below about 25 mg/L, or below about 10 mg/L. The accumulation of indole can be controlled by balancing enzyme expression (and in particular, balancing the upstream and downstream pathways) and activity using the multivariate modular approach as described in U.S. Pat. No. 8,927,241 (which is hereby incorporated by reference). In some embodiments, the accumulation of indole is controlled by chemical means. Other markers for efficient production of terpene and terpenoids, include accumulation of DOX or ME in the culture media. As described in U.S. Pat. No. 10,480,015, which is hereby incorporated by reference in its entirety, the bacterial strains can be constructed to avoid accumulation of large amounts of these chemical species, which accumulate in the culture at less than about 5 g/L, or less than about 4 g/L, or less than about 3 g/L, or less than about 2 g/L, or less than about 1 g/L, or less than about 500 mg/L, or less than about 100 mg/L. In some embodiments, MEcPP is the predominant MEP metabolite in the culture media, although its accumulation is limited by the genetic modifications to the bacterial strain, which pull MEP carbon downstream to IPP and DMAPP precursors. In various embodiments, MEcPP accumulates in the culture at less than about 30 g/L, or less than about 20 g/L, or less than about 2 g/L, or less than about 1 g/L, or less than about 500 mg/L, or less than about 100 mg/L.

The optimization of terpene or terpenoid production by manipulation of MEP pathway genes, as well as manipulation of the upstream and downstream pathways, is not expected to be a simple linear or additive process. Rather, through combinatorial analysis, optimization is achieved through balancing components of the MEP pathway, as well as upstream and downstream pathways. Indole accumulation (including prenylated indole) and MEP metabolite accumulation (e.g., DOX, ME, MEcPP, HIVIBPP, farnesol, prenol and isoprenol) in the culture or cells can be used as surrogate markers to guide this process.

Product can be recovered by any suitable process. Generally, recovery includes separation of material comprising product from the culture or cells, followed by extraction and purification. For example, recovery of some products can include partitioning the desired product into an organic phase or hydrophobic phase. Alternatively, the aqueous phase can be recovered, or the whole cell biomass can be recovered, for further processing. For example, in some embodiments, the product is a volatile product, which can include certain terpenoids. In such embodiments, the product can be recovered from an organic or hydrophobic phase that is mechanically separated from the culture. Alternatively or in addition, the product is harvested from the liquid and/or solid phase. In some embodiments, the product is purified by sequential extraction and purification. For example, the product may be purified by chromatography-based separation and recovery, such as supercritical fluid chromatography. The product may be purified by distillation, including simple distillation, steam distillation, fractional distillation, wipe-film distillation, or continuous distillation.

In some embodiments, the product is a non-volatile product, which in some embodiments is an extracellular product recovered from the culture medium. Alternatively, the product is an intracellular product recovered from harvested cell material. Where the product is poorly soluble, it may be recovered by filtration, and optionally with solvent extraction (e.g., extraction with ethanol). Alternatively, or in addition, the product is recovered by chromatography-based separation, such as liquid chromatography. In some embodiments, the product is recovered by sequential extraction and purification. In still other embodiments, the product is crystallized or precipitated out of solution.

The production of the desired product can be determined and/or quantified, for example, by gas chromatography (e.g., GC-MS). Production of product, recovery, and/or analysis of the product can be done as described in US 2012/0246767, which is hereby incorporated by reference in its entirety. For example, in some embodiments, product oil is extracted from aqueous reaction medium using an organic solvent, such as an alkane such as heptane or dodecane, followed by fractional distillation. In other embodiments, product oil is extracted from aqueous reaction medium using a hydrophobic phase, such as a vegetable oil, followed by organic solvent extraction and fractional distillation (see WO 2020/072908, which is hereby incorporated by reference in its entirety). Components of fractions may be measured quantitatively by GC/MS, followed by blending of fractions to generate a desired product profile.

Other embodiments of the invention will be apparent from the following working examples.

### EXAMPLES

### Example 1: Engineering of a Robust Escherichia coli Chassis Strain for Large-Scale Fermentations

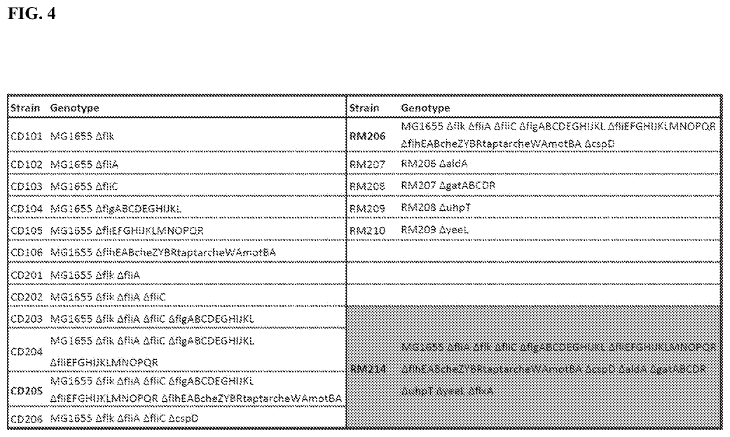

A series of deletion strains were constructed from E. coli MG1655 aiming for a robust phenotype in large-scale applications. MG1655 is a wild type strain. Deletion targets were selected with high add-on to maintenance, and which are aberrantly expressed when cells are exposed to nutrient heterogeneities. All deletions were conducted on the premise of strict neutrality towards growth parameters in glucose minimal medium. A list of deletion strains and their genotypes are shown in FIG. 4. The final strain of the series, named E. coli RM214, had the genotype:
ΔfliAΔflkΔfliCΔflgABCDEGHIJKLΔfliEFGHIJKLMNOPQRΔflhEABcheZYBRtapta rcheWAmotBAΔcspDAaldAΔgatABCDRΔuhpTΔyeeLΔflxΔ.

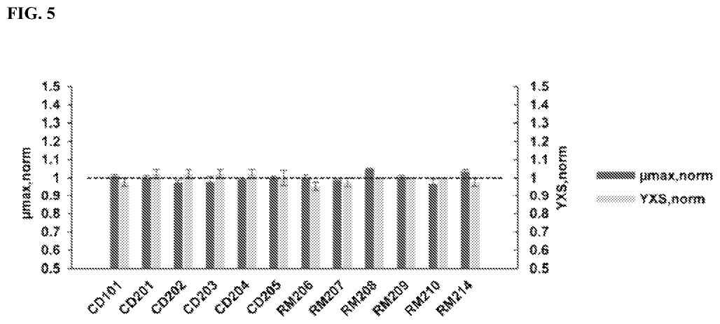

As shown in FIG. 5, the deletion strains did not exhibit any impacts on growth phenotype. MG1655 had the following values in the medium used: μₘₐₓ (l/h)=0.542+/−0.014; YXS (g/g)=0.454+1-0.013.

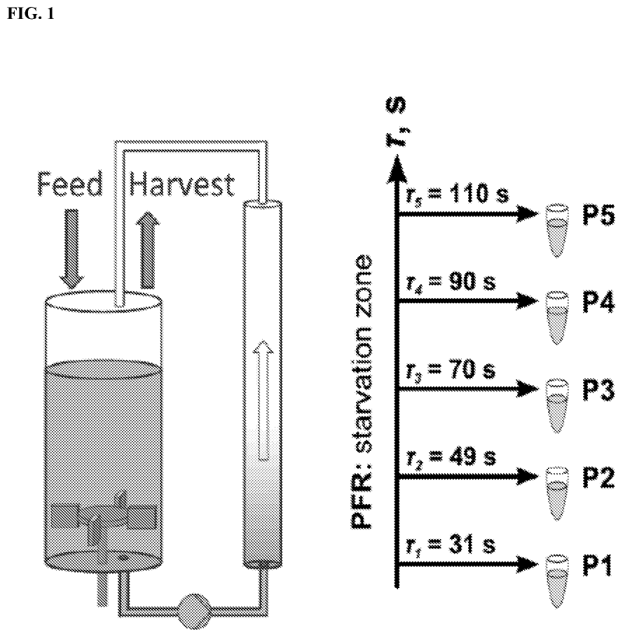

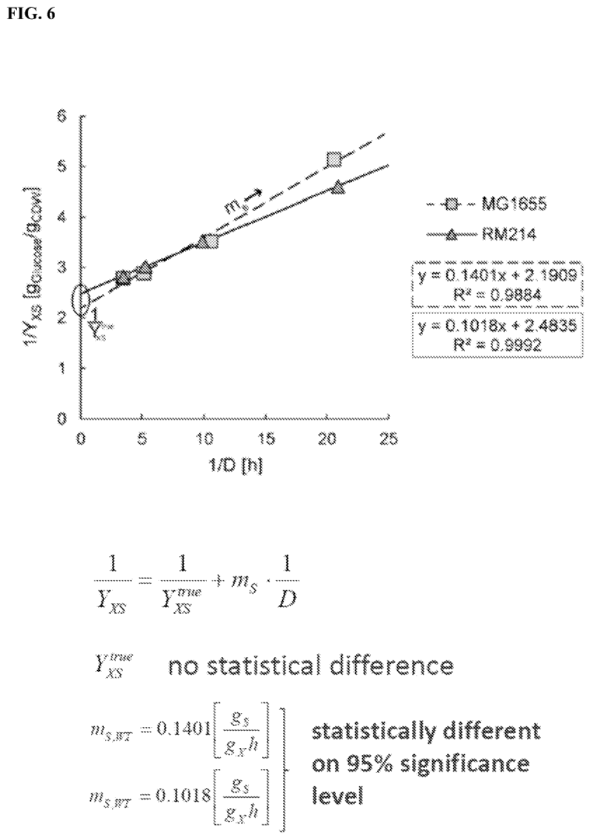

RM214 was fermented in continuous cultivations (D=0.2 h⁻¹) in an STR-PFR scale-down reactor, essentially as described in Löffler M, et al., Metabolic Engineering 38 (2016) 73-85. An STR-PFR (Stirred tank reactor-plug flow reactor) is illustrated in FIG. 1. The PFG imposes gradients that simulate large-scale conditions. The scale-down reactor system simulated repeated passages through a glucose starvation zone designed to be representative of passaging through nutrient poor zones in large-scale reactors. When exposed to nutrient gradients, E. coli RM214 had a significantly lower maintenance coefficient than E. coli MG1655 (mₛ=0.10 g_{Glucose}/g_{CDW}/h for RM214 vs mₛ=0.14 g_{Glucose}/g_{CDW}/h for MG1655, p<0.05). FIG. 6. In an exemplary STR-PFR protein production scenario, both strains exhibited similar biomass specific eGFP content in well-mixed STR conditions (0.140±0.004 g_{eGFp}/g_{CDW} for RM214 and 0.137±0.006 g_{eGFP}/g_{CDW} for MG1655).

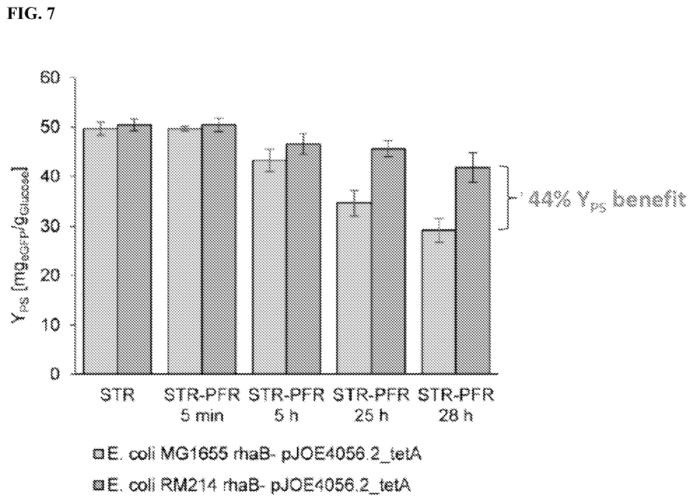

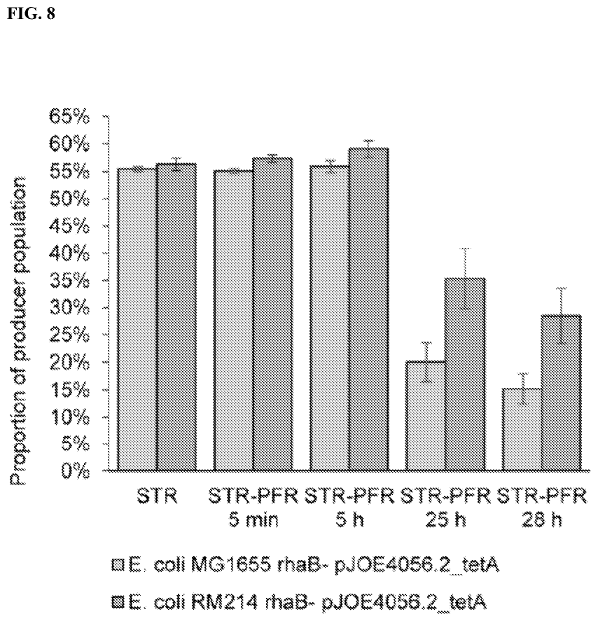

When exposed to STR-PFR conditions, E. coli RM214 remained significantly more productive reaching a final cellular eGFP content 49% higher than E. coli MG1655 after 28 h (44% improvement in mg_{eGFp}/g_{glucose}). FIG. 7. Based in FACS analysis, RM214 also showed a significantly larger fraction of eGFP producers at 25 and 28 hours. FIG. 8.

### Example 2: Challenging E. coli SR Under Simulated Large Scale Conditions

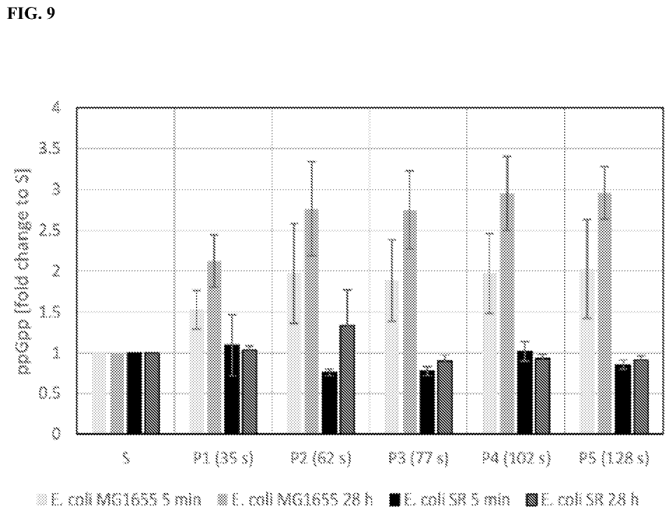

E. coli strain SR (MG1655 with ΔrelA, spotT[R290E; K292D]), which has an impaired stringent response, was tested using STR-PFR. ppGpp was quantified at P1 (35 sec.), P2 (62 sec.), P3 (77 sec.), P4 (102 sec.), and P5 (128 sec.) and compared against STR ppGpp (S). ppGpp was quantified after culture for 5 min. and 28 hours. In all cases, E. coli SR produces substantially less ppGpp, and production of ppGpp is similar to S conditions. FIG. 9.

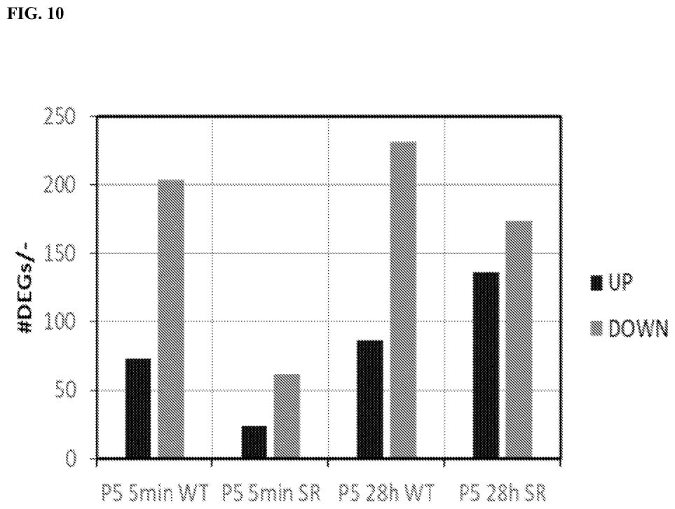

As shown in FIG. 8, E. coli SR produced less differentially expressed genes (DEGs) in the short term (P5 outlet of PFR at 5 minutes), showing a dampened short term response. FIG. 10. The long term response (P5 outlet of PFR at 28 hours) was fuzzy, with more upregulated transcripts and fewer downregulated transcripts than WT).

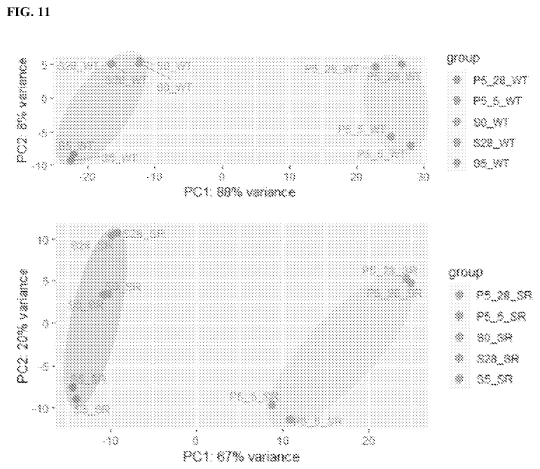

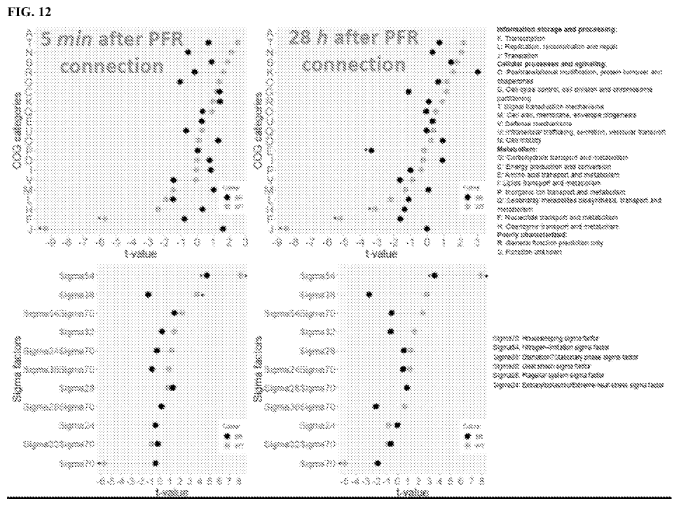

FIG. 11 shows 2-dimensional principal component analysis of total transcripts measured in the stirred tank reactor after connection with PFR. Inside PFR, nitrogen limitation was repeatedly imposed on E. coli Wildtype (WT) and on E. coli SR, a stringent response deficient strain. FIG. 12 shows differentially expressed genes of E. coli WT and E. coli SR grouped in COG categories and measured after frequent exposure to nitrogen limitation inside PFR (Top). Assignment of said gene transcripts to sigma factors is also shown (Bottom).

SEQUENCES
SEQ ID NO: 1: Zymomonas mobilis glf
MSSESSQGLVTRLALIAAIGGLLFGYDSAVIAAIGTPVDIHFIAPRHLSA
TAAASLSGMVVVAVLVGCVTGSLLSGWIGIRFGRRGGLLMSSICFVAAGF
GAALTEKLEGTGGSALQIFCFFRFLAGLGIGVVSTLTPTYIAEIAPPDKR
GQMVSGQQMAIVTGALTGYIFTWLLAHFGSIDWVNASGWCWSPASEGLIG
IAFLLLLLTAPDTPHWLVMKGRHSEASKILARLEPQADPNLTIQKIKAGE
DKAMDKSSAGLFAFGITVVFAGVSVAAFQQLVGINAVLYYAPQMFQNLGF
GADTALLQTISIGVVNFIFTMIASRVVDREGRKPLLIWGALGMAAMMAVL
GCCFWFKVGGVLPLASVLLYIAVFGMSWGPVCWVVLSEMFPSSIKGAAMP
IAVTGQWLANILVNFLFKVADGSPALNQTENHGESYLVFAALSILGGLIV
ARFVPETKGRSLDEIEEMWRSQK
SEQ ID NO: 2: Zymomonas mobilis glk
MEIVAIDIGGTHARFSIAEVSNGRVLSLGEETTEKTAEHASLQLAWERFG
EKLGRPLPRAAAIAWAGPVHGEVLKLTNNPWVLRPATLNEKLDIDTHVLI
NDFGAVAHAVAHMDSSYLDHICGPDEALPSDGVITILGPGTGLGVAHLLR
TEGRYFVIETEGGHIDFAPLDRLEDKILARLRERFRRVSIERIISGPGLG
NIYEALAAIEGVPESLLDDIKLWQMALEGKDNLAEAALDRFCLSLGAIAG
DLALAQGATSVVIGGGVGLRIASHLPESGFRQRFVSKGRFERVMSKIPVK
LITYPQPGLLGAAAAYANKYSEVE
SEQ ID NO: 3: E. coli Ydbk
MITIDGNGAVASVAFRTSEVIAIYPITPSSTMAEQADAWAGNGLKNVWGD
TPRVVEMQSEAGAIATVHGALQTGALSTSFTSSQGLLLMIPTLYKLAGEL
TPFVLHVAARTVATHALSIFGDHSDVMAVRQTGCAMLCAANVQEAQDFAL
ISQIATLKSRVPFIHFEDGERTSHEINKIVPLADDTILDLMPQVEIDAHR
ARALNPEHPVIRGTSANPDTYFQSREATNPWYNAVYDHVEQAMNDESAAT
GRQYQPFEYYGHPQAERVIILMGSAIGTCEEVVDELLTRGEKVGVLKVRL
YRPFSAKHLLQALPGSVRSVAVLDRTKEPGAQAEPLYLDVMTALAEAFNN
GERETLPRVIGGRYGLSSKEFGPDCVLAVFAELNAAKPKARFTVGIYDDV
TNLSLPLPENTLPNSAKLEALFYGLGSDGSVSATKNNIKIIGNSTPWYAQ
GYFVYDSKKAGGLTVSHLRVSEQPIRSAYLISQADFVGCHQLQFIDKYQM
AERLKPGGIFLLNTPYSADEVWSRLPQEVQAVLNQKKARFYVINAAKIAR
ECGLAARINTVMQMAFFHLTQILPGDSALAELQGAIAKSYSSKGQDLVER
NWQALALARESVEEVPLQPVNPHSANRPPVVSDAAPDFVKTVTAAMLAGL
GDALPVSALPPDGTWPMGTTRWEKRNIAEEIPIWKEELCTQCNHCVAACP
HSAIRAKVVPPEAMENAPASLHSLDVKSRDMRGQKYVLQVAPEDCTGCNL
CVEVCPAKDRQNPEIKAINMMSRLEHVEEEKINYDFFLNLPEIDRSKLER
IDIRTSQLITPLFEYSGACSGCGETPYIKLLTQLYGDRMLIANATGCSSI
YGGNLPSTPYTTDANGRGPAWANSLFEDNAEFGLGFRLTVDQHRVRVLRL
LDQFADKIPAELLTALKSDATPEVRREQVAALRQQLNDVAEAHELLRDAD
ALVEKSIWLIGGDGWAYDIGFGGLDHVLSLTENVNILVLDTQCYSNTGGQ
ASKATPLGAVTKFGEHGKRKARKDLGVSMMMYGHVYVAQISLGAQLNQTV
KAIQEAEAYPGPSLIIAYSPCEEHGYDLALSHDQMRQLTATGEWPLYRED
PRRADEGKLPLALDSRPPSEAPEETLLHEQRFRRLNSQQPEVAEQLWKDA
AADLQKRYDFLAQMAGKAEKSNTD
SEQ ID NO: 4: Saccharomyces cerevisiae choline
kinase
MVQESRPGSVRSYSVGYQARSRSSSQRRHSLTRQRSSQRLIRTISIESDV
SNITDDDDLRAVNEGVAGVQLDVSETANKGPRRASATDVTDSLGSTSSEY
IEIPFVKETLDASLPSDYLKQDILNLIQSLKISKWYNNKKIQPVAQDMNL
VKISGAMTNAIFKVEYPKLPSLLLRIYGPNIDNIIDREYELQILARLSLK
NIGPSLYGCFVNGRFEQFLENSKTLTKDDIRNWKNSQRIARRMKELHVGV
PLLSSERKNGSACWQKINQWLRTIEKVDQWVGDPKNIENSLLCENWSKEM
DIVDRYHKWLISQEQGIEQVNKNLIFCHNDAQYGNLLFTAPVMNTPSLYT
APSSTSLTSQSSSLFPSSSNVIVDDIINPPKQEQSQDSKLVVIDFEYAGA
NPAAYDLANHLSEWMYDYNNAKAPHQCHADRYPDKEQVLNFLYSYVSHLR
GGAKEPIDEEVQRLYKSIIQWRPTVQLFWSLWAILQSGKLEKKEASTAIT
REEIGPNGKKYIIKTEPESPEEDFVENDDEPEAGVSIDTEDYMAYGRDKI
AVFWGDLIGLGIITEEECKNESSFKELDTSYL

1. A genome-modified bacterial strain comprising deletions or inactivations of at least ten non-essential genes coding for two or more of: flagella components or transcriptional regulators thereof, chemotaxis proteins and regulators thereof, inhibitors of DNA replication, and proteins involved in active transport of sugars other than glucose; the strain exhibiting advantages in maintenance and biosynthesis performance under large-scale conditions as compared to a parent strain that does not contain said deletions.

2. The bacterial strain of claim 1, wherein the bacterial strain is a species of Escherichia, Bacillus, Corynebacterium, Pseudomonas, Rhodobacter, Zymomonas, Lactococcus, and Streptomyces.

3. The bacterial strain of claim 2, wherein the bacterial strain is Escherichia coli, Bacillus subtilis, Corynebacterium glutamicum, Pseudomonas putida, Pseudomonas chlororaphis, Rhodobacter capsulatus, Rhodobacter sphaeroides, Zymomonas mobilis, Lactococcus lactis, and Streptomyces coelicolor.

4. The bacterial strain of claim 3, wherein the bacterial strain is Escherichia coli.

5. The bacterial strain of any one of claims 1 to 4, wherein the large-scale conditions are at least about 1000 L, or at least about 10,000 L, or at least about 40,000 L, or at least about 100,000 L.

6. The bacterial strain of any one of claims 1 to 5, wherein the strain has a deletion or inactivation of at least about 15 genes, at least about 20 genes, at least about 25 genes, at least about 30 genes, at least about 35 genes, or at least about 40 genes, or at least about 45 genes, or at least about 50 genes, or at least about 55 genes, or at least about 60 genes.

7. The bacterial strain of claim 6, wherein the genome size or transcriptional burden is reduced by at least about 1% from wild type.

8. The bacterial strain of any one of claims 1 to 7, wherein the strain has a deletion or inactivation of at least about 15 genes coding for two or more of flagella components or transcriptional regulators thereof, chemotaxis proteins and regulators thereof, inhibitors of DNA replication, and proteins involved in active transport of sugars.

9. The bacterial strain of claim 8, wherein the strain has a deletion or inactivation of at least about 20 genes coding for two or more of flagella components or transcriptional regulators thereof, chemotaxis proteins and regulators thereof, inhibitors of DNA replication, and proteins involved in active transport of sugars other than glucose.

10. The bacterial strain of claim 8, wherein the strain has a deletion or inactivation of at least about 25 genes coding for two or more of flagella components or transcriptional regulators thereof, chemotaxis proteins and regulators thereof, inhibitors of DNA replication, and proteins involved in active transport of sugars other than glucose.

11. The bacterial strain of claim 8, wherein the strain has a deletion or inactivation of at least about 30 genes coding for two or more of flagella components or transcriptional regulators thereof, chemotaxis proteins and regulators thereof, inhibitors of DNA replication, and proteins involved in active transport of sugars other than glucose.

12. The bacterial strain of claim 8, wherein the strain has a deletion or inactivation of at least about 35 genes coding for two or more of flagella components or transcriptional regulators thereof, chemotaxis proteins and regulators thereof, inhibitors of DNA replication, and proteins involved in active transport of sugars other than glucose.

13. The bacterial strain of claim 8, wherein the strain has a deletion or inactivation of at least about 40 genes coding for two or more of flagella components or transcriptional regulators thereof, chemotaxis proteins and regulators thereof, inhibitors of DNA replication, and proteins involved in active transport of sugars other than glucose.

14. The bacterial strain of claim 8, having a deletion or inactivation of at least 10 genes encoding flagella components or transcriptional regulators thereof.

15. The bacterial strain of claim 8, having a deletion or inactivation of at least 15 genes encoding flagella components or transcriptional regulators thereof.

16. The bacterial strain of claim 8, having a deletion or inactivation of at least 20 genes encoding flagella components or transcriptional regulators thereof.

17. The bacterial strain of claim 8, having a deletion or inactivation of at least 25 genes encoding flagella components or transcriptional regulators thereof.

18. The bacterial strain of claim 8, having a deletion or inactivation of at least 30 genes encoding flagella components or transcriptional regulators thereof.

19. The bacterial strain of any one of claims 14 to 18, wherein the bacterial strain is a strain of E. coli, and deleted or inactivated genes encoding flagella components or transcriptional regulators thereof are selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, and flhB.

20. The bacterial strain of claim 19, wherein the genes of the fliFGHIJK operon are deleted or inactivated and/or the genes of the fliMNOPQR operon are deleted or inactivated.

21. The bacterial strain of claim 19 or 20, wherein one or more of fliA, fliC, and/or fliE are deleted or inactivated.

22. The bacterial strain of any one of claims 19 to 21, wherein flk is deleted or inactivated.

23. The bacterial strain of any one of claims 19 to 22, wherein the genes of the flgBCDEGHIJ and/or flgKL operons are deleted or inactivated.

24. The bacterial strain of any one of claims 19 to 23, wherein genes of the flhBAE operon are deleted or inactivated.

25. The bacterial strain of any one of claims 19 to 24, having a deletion or inactivation of fliA, flk, fliC, flgABCDEGHIJKL, fliEFGHIJKLMNOPQR, and flhEAB.

26. The bacterial strain of any one of claims 19 to 25, wherein the strain has a deletion or inactivation of one or both of motB and motA.

27. The bacterial strain of any one of claims 1 to 26, wherein the strain has a deletion or inactivation of one or more genes encoding chemotaxis proteins and regulators thereof.

28. The bacterial strain of claim 27, wherein the strain has a deletion or inactivation of fliA.

29. The bacterial strain of claim 27 or 28, wherein the strain has a deletion or inactivation of one or more of tar, tap, cheR, cheB, cheY, cheZ, cheW, and cheA.

30. The bacterial strain of claim 29, wherein the strain has a deletion or inactivation of genes of the operon tar-tap-cheRBYZ.

31. The bacterial strain of claim 29 or 30, wherein the strain has a deletion or inactivation of genes of the operon motAB-cheAW.

32. The bacterial strain of any one of claims 1 to 31, wherein the strain has a deletion or inactivation of at least one gene encoding an inhibitor of DNA replication.

33. The bacterial strain of claim 32, wherein the strain has a deletion or inactivation of cspD.

34. The bacterial strain of any one of claims 1 to 33, wherein the strain has a deletion or inactivation of one or more genes involved in active transport of sugars, which are optionally phosphotransferase system (PTS) proteins.

35. The bacterial strain of claim 34, wherein the strain has a deletion or inactivation of one or more of gatA, gatB, gatC, gatD, gatR, and uhpT.

36. The bacterial strain of claim 35, wherein the strain has a deletion or inactivation of gatABCDR and uhpT.

37. The bacterial strain of any one of claims 1 to 36, further comprising a deletion or inactivation of one or more of aldA, yeeI, yeeL, and flxA.

38. The bacterial strain of claim 37, wherein the strain comprises at least 10 deletions or inactivations selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

39. The bacterial strain of claim 37, wherein the strain comprises at least 20 deletions or inactivations selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

40. The bacterial strain of claim 37, wherein the strain comprises at least 30 deletions or inactivations selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

41. The bacterial strain of claim 37, wherein the strain comprises deletions or inactivations of: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

42. The bacterial strain of any one of claims 1 to 41, wherein the strain is a strain of E. coli comprising deletions or inactivations selected from fliC, aroF, aldA, cstA, aceA, cspD, aceB, trg, groL, dnaK, yfiA, gatC, flgL, flgK, acs, mdh, kgtP, fliA, glnH, yjdA, rpoS, hupB, clpB, uhpT, clpA, yeeI, htpG, motA, argT, rplP, flxA, fadD, pheT, flgM, arcA, modA, leuD, fumA, yjcZ, gpmA, rpoE, rseA, maeB, katG, ychH, hns, ygaT, cheW, ybhQ, atpC, glyS, fadB, osmE, hcaR, uspE, uspA, dps, ygeV, pheA, yfiQ, ibpA, ihfB, livK, miaA, ihfA, cysA, bolA, ydcS, ucpA, uspF, nuoH, yeaA, yjjY, aldB, yeaG, araC, yqfA, grpE, aroP, sra, ygjG, cheY, acrB, tpx, cysN, yaeH, osmC, ugpB, ykfB, Smg, yfcY, hypD, elaB, rsd, ybaL, fadH, ubiA, ygaU, sseA, yccV, bfr, pheS, yihX, ytfQ, putP, cycA, yeaT, yiiU, ycgB, rrmJ, rob, agp, ybeL, lrp, ydhR, malP, fbaB, msyB, glcC, ydcJ, ubiC, lon, glpK, dacC, yqjC, glmM, ompR, selD, ygdH, trpA, mukB, nupG, gltD, uspG, yggG, yrbL, glcD, cfa, phoH, yniA, gst, mscL, yhiO, ybhL, yahO, yjgD, glpF, wbbK, yfcX, yqjD, paaJ, yqjE, ynjH, aroL, galS, fruA, mhpR, rnr, prlC, smf, ygaM, ynhG, glgC, csiE, yaiZ, ygiN, aphA, ivy, glgS, ybjP, puuD, udp, yceP, fxsA, ybbN, yhbT, yeaY, ybeZ, ycjG, erfK, yhaO, yjdC, astC, sbmC, yghZ, ydbJ, uxuA, yhaL, fucO, ygdR, yajO, elbB, yhdW, ytfJ, ygg, ycfP, yfbT, phoU, blc, pepT, yqcA, yegP, yghU, sfsA, ysgA, yohC, mgtA, ybbK, nhaA, adhP, yehZ, rmuC, ydeW, yqeC, yecA, mppA, treA, dkgA, malS, ddlB, yafY, exuR, yhjY, yfbU, nadR, ydgD, qor, slt, yjgB, yaiE, soxS, prpR, yeiE, yahK, ydjF, yjfO, talA, yodD, pmrD, yhdH, yliB, yliJ, yeeZ, yaeP, ybdK, msrA, ydhQ, rfaB, ybjQ, ygiE, grxB, yjjM, ydhF, ycbB, araF, ygjR, yqjG, cdaR, ymgE, glnG, deoC, ddpX, otsB, yhfZ, pheL, gmr, yibF, hdhA, ygdI, cysQ, yfdY, yceK, ydhC, yqjI, glpD, ydiZ, yhhA, yibT, ygjF, tam, yjfN, yacL, melR, yiaG, yjeF, ycfX, yiiT, kbl, dhaH, yafV, ycjX, ego, ytfR, sodC, ybaE, tdh, yhjD, fic, rpiB, ibpB, ytfN, yfiL, araB, relB, yadH, mrdB, ybiH, frwC, yjbR, psiF, basR, xylA, melA, ybhG, yhcO, garP, nlpE, ampE, ydaN, ydbC, pdxJ, glpT, fucA, yjbB, srlA, yddH, ybjK, yjgK, yadI, yncB, idi, sufA, zntR, ade, ynfD, yncG, yhcH, yhfY, yafM, yhhW, ybiI, yibI, ybeH, and ysaB.

43. The bacterial strain of any one of claims 1 to 42, comprising a deletion or inactivation of relA.

44. The bacterial strain of claim 43, further comprising a spoT mutant, which optionally comprises the amino acid substitutions R290E and K292D.

45. The bacterial strain of claim 43 or 44, further comprising an ace mutant, which optionally comprises a G267C substitution in aceE.

46. The bacterial strain of any one of claims 1 to 45, further comprising overexpression or complementation of a gene that increases glucose uptake.

47. The bacterial strain of claim 46, comprising recombinant expression of one or more of SEQ ID NO:1 and/or SEQ ID NO: 2, or derivatives thereof.

48. The bacterial strain of claim 47, wherein one or more phosphotransferase system (PTS) proteins are overexpressed.

49. The bacterial strain of any one of claims 1 to 48, wherein the strain has a maintenance coefficient of less than 0.14 g_{glucose}/g_{CDW}/h at large scale.

50. The bacterial strain of claim 49, wherein the strain has a maintenance coefficient of about 0.10 g_{glucose}/g_{CDW}/h or less at large scale.

51. The bacterial strain of any one of claims 1 to 50, wherein the strain produces a secondary metabolite selected from a terpenoid, flavonoid, alkaloid, cannabinoid, polyketide, stilbenoid, and polyphenol.

52. The bacterial strain of claim 51, wherein the strain has an overexpression of one or more MEP pathway genes.

53. The bacterial strain of claim 52, wherein the strain expresses a terpenoid biosynthesis pathway.

54. The bacterial strain of any one of claims 51 to 53, wherein the strain expresses at least one recombinant P450 enzyme.

55. The bacterial strain of any one of claims 51 to 54, wherein the strain expresses at least one uridine diphosphate-dependent glycosyl transferase (UGT) enzyme.

56. The bacterial strain of any one of claims 1 to 50, wherein the strain produces an antibiotic, amino acid, fatty acid, or nucleotide product.

57. The bacterial strain of any one of claims 1 to 50, wherein the strain produces a recombinant protein product or peptide.

58. The bacterial strain of claim 57, wherein the recombinant protein product is an industrial enzyme or therapeutic protein.

59. A genome-modified E. coli strain comprising at least 10 gene deletions or inactivations, including a plurality of deletions or inactivations selected from, rpoS, hupB, clpB, uhpT, clpA, yeeI, htpG, motA, argT, rplP, flxA, fadD, pheT, flgM, arcA, modA, leuD, fumA, yjcZ, gpmA, rpoE, rseA, maeB, katG, ychH, hns, ygaT, cheW, ybhQ, atpC, glyS, fadB, osmE, hcaR, uspE, uspA, dps, ygeV, pheA, yfiQ, ibpA, ihfB, livK, miaA, ihfA, cysA, bolA, ydcS, ucpA, uspF, nuoH, yeaA, yjjY, aldB, yeaG, araC, yqfA, grpE, aroP, sra, ygjG, cheY, acrB, tpx, cysN, yaeH, osmC, ugpB, ykfB, Smg, yfcY, hypD, elaB, rsd, ybaL, fadH, ubiA, ygaU, sseA, yccV, bfr, pheS, yihX, ytfQ, putP, cycA, yeaT, yiiU, ycgB, rrmJ, rob, agp, ybeL, lrp, ydhR, malP, fbaB, msyB, glcC, ydcJ, ubiC, lon, glpK, dacC, yqjC, glmM, ompR, selD, ygdH, trpA, mukB, nupG, gltD, uspG, yggG, yrbL, glcD, cfa, phoH, yniA, gst, mscL, yhiO, ybhL, yahO, yjgD, glpF, wbbK, yfcX, yqjD, paaJ, yqjE, ynjH, aroL, galS, fruA, mhpR, rnr, prlC, smf, ygaM, ynhG, glgC, csiE, yaiZ, ygiN, aphA, ivy, glgS, ybjP, puuD, udp, yceP, fxsA, ybbN, yhbT, yeaY, ybeZ, ycjG, erfK, yhaO, yjdC, astC, sbmC, yghZ, ydbJ, uxuA, yhaL, fucO, ygdR, yajO, elbB, yhdW, ytfJ, ygg, ycfP, yfbT, phoU, blc, pepT, yqcA, yegP, yghU, sfsA, ysgA, yohC, mgtA, ybbK, nhaA, adhP, yehZ, rmuC, ydeW, yqeC, yecA, mppA, treA, dkgA, malS, ddlB, yafY, exuR, yhjY, yfbU, nadR, ydgD, qor, slt, yjgB, yaiE, soxS, prpR, yeiE, yahK, ydjF, yjfO, talA, yodD, pmrD, yhdH, yliB, yliJ, yeeZ, yaeP, ybdK, msrA, ydhQ, rfaB, ybjQ, ygiE, grxB, yjjM, ydhF, ycbB, araF, ygjR, yqjG, cdaR, ymgE, glnG, deoC, ddpX, otsB, yhfZ, pheL, gmr, yibF, hdhA, ygdI, cysQ, yfdY, yceK, ydhC, yqjI, glpD, ydiZ, yhhA, yibT, ygjF, tam, yjfN, yacL, melR, yiaG, yjeF, ycfX, yiiT, kbl, dhaH, yafV, ycjX, ego, ytfR, sodC, ybaE, tdh, yhjD, fic, rpiB, ibpB, ytfN, yfiL, araB, relB, yadH, mrdB, ybiH, frwC, yjbR, psiF, basR, xylA, melA, ybhG, yhcO, garP, nlpE, ampE, ydaN, ydbC, pdxJ, glpT, fucA, yjbB, srlA, yddH, ybjK, yjgK, yadI, yncB, idi, sufA, zntR, ade, ynfD, yncG, yhcH, yhfY, yafM, yhhW, ybiI, yibI, ybeH, and ysaB.

60. The E. coli strain of claim 59, comprising deletion or inactivation of at least 5, at least 10, at least 15, at least 20, at least 25, or at least 30 genes selected from rpoS, hupB, clpB, uhpT, clpA, yeeI, htpG, motA, argT, rplP, flxA, fadD, pheT, flgM, arcA, modA, leuD, fumA, yjcZ, gpmA, rpoE, rseA, maeB, katG, ychH, hns, ygaT, cheW, ybhQ, atpC, glyS, fadB, osmE, hcaR, uspE, uspA, dps, ygeV, pheA, yfiQ, ibpA, ihfB, livK, miaA, ihfA, cysA, bolA, ydcS, ucpA, uspF, nuoH, yeaA, yjjY, aldB, yeaG, araC, yqfA, grpE, aroP, sra, ygjG, cheY, acrB, tpx, cysN, yaeH, osmC, ugpB, ykfB, Smg, yfcY, hypD, elaB, rsd, ybaL, fadH, ubiA, ygaU, sseA, yccV, bfr, pheS, yihX, ytfQ, putP, cycA, yeaT, yiiU, ycgB, rrmJ, rob, agp, ybeL, lrp, ydhR, malP, fbaB, msyB, glcC, ydcJ, ubiC, lon, glpK, dacC, yqjC, glmM, ompR, selD, ygdH, trpA, mukB, nupG, gltD, uspG, yggG, yrbL, glcD, cfa, phoH, yniA, gst, mscL, yhiO, ybhL, yahO, yjgD, glpF, wbbK, yfcX, yqjD, paaJ, yqjE, ynjH, aroL, galS, fruA, mhpR, rnr, prlC, smf, ygaM, ynhG, glgC, csiE, yaiZ, ygiN, aphA, ivy, glgS, ybjP, puuD, udp, yceP, fxsA, ybbN, yhbT, yeaY, ybeZ, ycjG, erfK, yhaO, yjdC, astC, sbmC, yghZ, ydbJ, uxuA, yhaL, fucO, ygdR, yajO, elbB, yhdW, ytfJ, ygg, ycfP, yfbT, phoU, blc, pepT, yqcA, yegP, yghU, sfsA, ysgA, yohC, mgtA, ybbK, nhaA, adhP, yehZ, rmuC, ydeW, yqeC, yecA, mppA, treA, dkgA, malS, ddlB, yafY, exuR, yhjY, yfbU, nadR, ydgD, qor, slt, yjgB, yaiE, soxS, prpR, yeiE, yahK, ydjF, yjfO, talA, yodD, pmrD, yhdH, yliB, yliJ, yeeZ, yaeP, ybdK, msrA, ydhQ, rfaB, ybjQ, ygiE, grxB, yjjM, ydhF, ycbB, araF, ygjR, yqjG, cdaR, ymgE, glnG, deoC, ddpX, otsB, yhfZ, pheL, gmr, yibF, hdhA, ygdI, cysQ, yfdY, yceK, ydhC, yqjI, glpD, ydiZ, yhhA, yibT, ygjF, tam, yjfN, yacL, melR, yiaG, yjeF, ycfX, yiiT, kbl, dhaH, yafV, ycjX, ego, ytfR, sodC, ybaE, tdh, yhjD, fic, rpiB, ibpB, ytfN, yfiL, araB, relB, yadH, mrdB, ybiH, frwC, yjbR, psiF, basR, xylA, melA, ybhG, yhcO, garP, nlpE, ampE, ydaN, ydbC, pdxJ, glpT, fucA, yjbB, srlA, yddH, ybjK, yjgK, yadI, yncB, idi, sufA, zntR, ade, ynfD, yncG, yhcH, yhfY, yafM, yhhW, ybiI, yibI, ybeH, and ysaB.

61. The E. coli strain of claim 59 or 60, further comprising a deletion or inactivation of one or more of fliC, aroF, aldA, cstA, aceA, cspD, aceB, trg, groL, dnaK, yfiA, gatC, flgL, flgK, acs, mdh, kgtP, fliA, glnH, and yjdA.

62. The E. coli strain of any one of claims 59 to 61, wherein the genome size or transcriptional burden of native genes is reduced by at least about 1% from wild type.

63. The E. coli strain of any one of claims 59 to 62, wherein at least five deleted or inactivated genes are selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, and flhB.

64. The E. coli strain of claim 63, wherein the genes of the fliFGHIJK operon are deleted or inactivated and/or the genes of the fliMNOPQR operon are deleted or inactivated.

65. The E. coli strain of claim 63 or 64, wherein one or more of fliA, fliC, and/or fliE are deleted or inactivated.

66. The E. coli strain of any one of claims 59 to 65, wherein flk is deleted or inactivated.

67. The E. coli strain of any one of claims 59 to 66, wherein the genes of the flgBCDEGHIJ and/or flgKL operons are deleted or inactivated.

68. The E. coli strain of any one of claims 59 to 67, wherein genes of the flhBAE operon are deleted or inactivated.

69. The E. coli strain of claim 68, comprising a deletion or inactivation of fliA, flk, fliC, flgABCDEGHIJKL, fliEFGHIJKLMNOPQR, and flhBAE.

70. The E. coli strain of any one of claims 59 to 69, wherein the strain comprises a deletion or inactivation of one or both of motB and motA.

71. The E. coli strain of any one of claims 59 to 70, wherein the strain comprises a deletion or inactivation of one or more of tar, tap, cheR, cheB, cheY, cheZ, cheW, and cheA.

72. The E. coli strain of claim 71, wherein the strain has a deletion or inactivation of genes of the operon tar-tap-cheRBYZ.

73. The E. coli strain of any one of claims 70 to 72, wherein the strain comprises a deletion or inactivation of genes of the operon motAB-cheAW.

74. The E. coli strain of any one of claims 59 to 73, wherein the strain has a deletion or inactivation of cspD.

75. The E. coli strain of any one of claims 59 to 74, wherein the strain comprises a deletion or inactivation of one or more of gatA, gatB, gatC, gatD, gatR, and uhpT.

76. The E. coli strain of claim 75, wherein the strain comprises a deletion or inactivation of gatABCDR and uhpT.

77. The E. coli strain of any one of claims 59 to 76, further comprising a deletion or inactivation of one or more of aldA, yeeI, yeeL, and flxA.

78. The E. coli strain of claim 59, wherein the strain comprises at least 10 deletions or inactivations selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

79. The E. coli strain of claim 59, wherein the strain comprises at least 20 deletions or inactivations selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

80. The E. coli strain of claim 59, wherein the strain comprises at least 30 deletions or inactivations selected from: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

81. The E. coli strain of claim 59, wherein the strain comprises deletions or inactivations of: fliA, flk, fliC, flgA, flgB, flgC, flgD, flgE, flgG, flgH, flgI, flgJ, flgK, flgL, fliE, fliF, fliG, fliH, fliI, fliJ, fliK, fliL, fliM, fliN, fliO, fliP, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

82. The E. coli strain of any one of claims 59 to 81, comprising a deletion or inactivation of relA.

83. The E. coli strain of claim 82, further comprising a spoT mutant, which optionally comprises the amino acid substitutions R290E and K292D.

84. The E. coli strain of claim 82 or 83, further comprising an ace mutant, which optionally comprises a G267C substitution in aceE.

85. The E. coli strain of any one of claims 59 to 84, further comprising overexpression or complementation of a gene that increases glucose uptake.

86. The E. coli strain of claim 85, comprising recombinant expression of one or more of SEQ ID NO:1 and/or SEQ ID NO: 2, or derivatives thereof.

87. The E. coli strain of any one of claims 59 to 85, wherein the strain has a maintenance coefficient of less than 0.14 g_{glucose}/g_{CDW}/h at large scale.

88. The E. coli strain of claim 87, wherein the strain has a maintenance coefficient of about 0.10 g_{glucose}/g_{CDW}/h or less at large scale.

89. The E. coli strain of any one of claims 59 to 88, wherein the strain produces a secondary metabolite selected from a terpenoid, flavonoid, alkaloid, cannabinoid, polyketide, stilbenoid, and polyphenol.

90. The E. coli strain of claim 89, wherein the strain has an overexpression of one or more MEP pathway genes.

91. The E. coli strain of claim 90, wherein the strain expresses a terpenoid biosynthesis pathway or an Isopentenol Utilization Pathway.

92. The E. coli strain of any one of claims 89 to 91, wherein the strain expresses at least one recombinant P450 enzyme.

93. The E. coli strain of any one of claims 59 to 92, wherein the strain expresses at least one uridine diphosphate-dependent glycosyl transferase (UGT) enzyme, and optionally comprises one or more genetic modifications to increase UDP-glucose availability.

94. The E. coli strain of any one of claims 59 to 88, wherein the strain produces a recombinant protein product or peptide.

95. The E. coli strain of claim 94, wherein the recombinant protein product is an industrial enzyme, plant protein, or therapeutic protein.

96. The E. coli strain of any one of claims 59 to 88, wherein the strain produces an antibiotic, amino acid, fatty acid, or nucleotide product.

97. A method for making a product by large scale fermentation, comprising, culturing the strain of any one of claims 1 to 96 at large scale.

98. The method of claim 97, wherein the large-scale is at least about 1000 L, or at least about 10,000 L, or at least about 40,000 L, or at least about 100,000 L.

99. The method of claim 97 or 98, wherein the culture is batch culture, continuous culture, or semi-continuous culture.

100. The method of claim 99, wherein the culture is conducted with a stirred tank reactor or bubble column reactor.

101. The method of claim 99 or 100, wherein the culture is a fed-batch process comprising a first phase where bacterial biomass is created, followed by a production phase.

102. The method of claim 101, wherein the production phase is nutrient limited.

103. The method of claim 101 or 102, wherein the production phase is about 24 hours or more, or about 36 hours or more, or about 48 hours or more, or about 60 hours or more, or about 72 hours or more.

104. The method of any one of claims 97 to 103, wherein the bacterial strain is cultured at a temperature from about 22° C. to about 37° C.

105. The method of claim 104, wherein the culturing is conducted at about 25° C. or greater, about 30° C. or greater, about 32° C. or greater, or about 34° C. or greater, or about 37° C. in some embodiments.

106. The method of any one of claims 97 to 105, further comprising recovering the product from the cell culture or from cell lysates.
